(19) United States
(12) Patent Application Publication
(10) Pub. No.: US 2011/0178420 A1
(43) Pub. Date: Jul. 21, 2011

(54) METHODS AND APPARATUSES FOR IMPROVING BREATH ALCOHOL TESTING

(76) Inventors: Trent Ridder, Woodbridge, VA (US); Bill Kardeen, Albuquerque, NM (US); Bentley Laaksonen, Albuquerque, NM (US); Ben Ver Steeg, Redlands, CA (US); James McNally, Albuquerque, NM (US); Mike Mills, Tijeras, NM (US)

(21) Appl. No.: 13/008,000

(22) Filed: Jan. 17, 2011

Related U.S. Application Data
(60) Provisional application No. 61/295,825, filed on Jan. 18, 2010.

(51) Int. Cl. A61B 5/08

(52) U.S. Cl. 600/532

(57) ABSTRACT

Some embodiments of the present invention provide methods and apparatuses for improving the performance and utility of breath alcohol measurements through the use of multivariate spectroscopy. In some embodiments, the spectroscopic breath measurement can be combined with multivariate spectroscopic tissue alcohol and/or tissue biometric measurements in order to overcome the limitations encountered by existing breath alcohol measurement devices.

### CROSSREFERENCE TO RELATED APPLICATIONS

This application claims priority to U.S. provisional 61/295,825, filed Jan. 18, 2010, which is incorporated herein by reference.

### TECHNICAL FIELD

The present invention relates to improvements to measuring the presence or concentration of alcohol using breath-based approaches. The present invention further relates to monitoring for the presence or concentration of alcohol or other substances in individuals in probation/corrections, alcohol treatment centers, hospitals, vehicles, law enforcement, and restricted access environments, and more specifically to methods and apparatuses for detecting the presence or concentration of alcohol or substances of abuse in individuals in any of a variety of controlled environments using breath based approaches.

### BACKGROUND OF THE INVENTION

Alcohol abuse is a national problem that extends into virtually all aspects of society. Current practice for alcohol measurements to detect alcohol abuse is typically based upon either blood measurements or breath testing. Blood measurements are generally considered the “gold standard” for determining alcohol intoxication levels. However, blood measurements typically require either a venous or capillary sample and involve significant handling precautions in order to minimize health risks. Once extracted, the blood sample must be properly labeled and transported to a clinical laboratory or other suitable location where a clinical gas chromatograph is typically used to measure the blood alcohol level. Due to the invasiveness of the procedure and the amount of sample handling involved, blood alcohol measurements are usually limited to critical situations such as for traffic accidents, violations where the suspect requests this type of test, and accidents where injuries are involved.

Because it is less invasive, breath testing is more commonly encountered in the field. In breath testing, the subject must expire air into the instrument for a sufficient time and volume to achieve a stable breath flow that originates from the alveoli deep within the lungs. The device then measures the alcohol content in the air, which is related to blood alcohol through a blood-breath ratio (BBR). The blood-breath ratio used in the United States is 2100 and varies between 1900 and 2400 in other nations. The variability in the BBR is due to the fact that it is dependent on each person's physiology. In other words, each subject will generally have a BBR in the 1900 to 2400 range depending on his or her physiology. Since knowledge of each subject's BBR is unavailable in field applications, each nation assumes a single partition coefficient value that is globally applied to all measurements. In the U.S., defendants in DUI cases often use the globally applied BBR as an argument to impede prosecution.

Currently available breath measurements have additional limitations. First, the presence of “mouth alcohol” can falsely elevate the breath alcohol measurement. This necessitates a 15-minute waiting period prior to making a measurement in order to ensure that no mouth alcohol is present. For a similar reason, a 15 minute delay is required for individuals who are observed to burp or vomit. A delay of 10 minutes or more is often required between breath measurements to allow the instrument to return to equilibrium with the ambient air and zero alcohol levels.

The disadvantages of the breath BBR and mouth alcohol issues can be greatly alleviated by incorporating a non-breath alcohol test. In some embodiments of the present invention, a tissue alcohol measurement is used in conjunction with a breath alcohol measurement in order to detect situations where either of the alcohol results is suspect. In cases where both measurements are in agreement, the likelihood of BBR or mouth alcohol errors is greatly reduced which can eliminate many of the arguments that are presently used to impede prosecution.

In addition, the accuracy of breath alcohol measurements is sensitive to numerous physiological and environmental factors including airborne chemical interferents such as acetone, isopropanol, carbon dioxide, and methyl ethyl ketone that can yield alcohol concentration errors. Many evidential breath testers use infrared (IR) spectroscopy to perform the alcohol assay. Currently available embodiments of IR breath testers use between 1 and 4 wavelengths of IR radiation to perform the alcohol measurement. However, full-spectrum IR measurements can be performed that can provide spectra containing hundreds of wavelengths. This additional information can be used to significantly reduce or eliminate errors associated with spectrometer or environmentally related drift as well as errors arising from the presence of chemical interferents in the air.

Another concern for breath tests is that they typically require a means for verifying that the test is being performed on the desired individual. In some environments, such as law enforcement, this is not a drawback as a law enforcement official is already present to administer the test. In other environments, such as home arrest, a means for verifying the identity of the person being tested without the need for a test administrator to be present would be advantageous. Some embodiments of the present invention provide methods and apparatuses incorporating quantitative spectroscopy that improve breath alcohol tests by addressing the concerns regarding the BBR, mouth alcohol events, chemical and environmental interferents, and verification of the identity of the person being tested.

### SUMMARY OF THE INVENTION

Some embodiments of the present invention provide methods and apparatuses for improving the performance and utility of breath alcohol measurements through the use of multivariate spectroscopy. In some embodiments, the spectroscopic breath measurement can be combined with multivariate spectroscopic tissue alcohol and/or tissue biometric measurements in order to overcome the limitations encountered by existing breath alcohol measurement devices. For demonstrative purposes the discussion herein generally refers to infrared and near-infrared spectroscopic measurements, however, visible (UV-vis), Raman, and fluorescence spectroscopic measurements are also suitable for use in the present invention.

Absorption spectroscopy is a generally known analytical method. In some forms, absorption spectroscopy measures the electromagnetic radiation (typical wavelength range of 0.3-25 μm) that a substance absorbs at various wavelengths, though other methods measure other effects a substance has on incident light. Absorption phenomena can be related to molecular vibrations and shifts in energy levels of individual atoms or electrons within a molecule. These phenomena cause the absorbing molecule or atom to switch to a higher energy state. Absorption occurs most frequently in limited ranges of wavelengths that are based upon the molecular structure of the species present in the measured sample. Thus, for light at several wavelengths passing through a substance, the substance will absorb a greater percentage of photons at certain wavelengths than it will at others.

At the molecular level, many primary vibrational transitions occur in the mid-infrared wavelength region (i.e., wavelengths between 2.5-6 μm). However, for some measurements, use of the mid-infrared region can be problematic because molecules with strong absorbance properties (e.g., water) can result in the total absorption of virtually all light introduced to the sample being measured. The problem can often be overcome through the use of shorter wavelengths (typically in the near infrared region of 0.7-2.5 μm) where weaker overtones and combinations of the mid-infrared vibrations exist. Thus, the near-infrared region can be employed in such situations as it preserves the qualitative and quantitative properties of mid-infrared measurements while helping to alleviate the problem of total light absorption.

As mentioned above, alcohol and other analytes absorb light at multiple wavelengths in both the mid- and near-infrared range. Due to the overlapping nature of these absorption bands, reliable analyte measurements can be very difficult if only a single wavelength is used for analysis. Thus, analysis of spectral data can incorporate absorption characteristics at several wavelengths, which enables sensitive and selective measurements of the desired analytes. In multi-wavelength spectroscopy, multivariate analysis techniques can be used to empirically determine the relationship between measured spectra and a property of interest (e.g., analyte concentration). A significant advantage of the present invention is that, because different analytes exhibit different absorption spectra, multivariate spectroscopy can be used to perform multiple analyte or property measurements simultaneously. This can be performed using a single spectroscopic breath measurement (e.g. measure multiple analytes or properties in breath) or in conjunction with another spectroscopic measurement such as that from tissue (e.g. one or more analyte or property measurements in each of the breath and tissue measurements). There are a variety of potential analytes and properties that are of interest in the present invention that include, but are not limited to: alcohol, alcohol byproducts, alcohol adducts, biometric properties or attributes, or substances of abuse.

The advantages and features of novelty that characterize the present invention are pointed out with particularity in the claims annexed hereto and forming a part hereof. However, for a better understanding of the invention, reference should be made to the drawings which form a further part hereof, and to the accompanying descriptive matter in which there are illustrated and described embodiments of the present invention.

Example embodiments of the present invention are disclosed herein. However, it is to be understood that the disclosed embodiments are merely exemplary of the present invention that can be embodied in various systems. Therefore, specific details disclosed herein are not to be interpreted as limiting, but rather as a basis for the claims and as a representative basis for teaching one of skill in the art to variously practice the invention.

### BRIEF DESCRIPTION OF THE DRAWINGS

The accompanying drawings, which are incorporated in and form part of the specification, illustrate the present invention and, together with the description, describe the invention. In the drawings, like elements are referred to by like numbers.

FIG. 1 is a graph of 16 cm⁻¹ near-infrared spectra of ethanol, isopropyl alcohol, methanol, acetone, toluene, methyl ethyl ketone (MEK), and chlorobenzene obtained from a Fourier Transform spectrometer.

FIG. 2 is an illustration of CLS concentration estimates versus known concentrations for the 7 analyte mixtures.

FIG. 3 is an illustration of the Net Analyte Signal (NAS) for a 3 component system.

FIG. 4 is an illustration of PLS concentration estimates versus known concentrations for the 7 analyte mixtures.

FIG. 5 is a schematic illustration of a multivariate spectroscopic breath device in accord with the present invention.

FIG. 6 is a schematic illustration of a multivariate spectroscopic breath device in accord with the present invention.

FIG. 7 is a schematic illustration of a multivariate spectroscopic breath device with combined light source and spectrometer in accord with the present invention.

FIG. 8 is a listing of substances known to be breath alcohol interferents.

FIG. 9 is a plot of breath versus blood alcohol concentration acquired from a clinical study.

FIG. 10 is a plot of tissue versus blood alcohol concentration acquired from the same clinical study as in FIG. 9.

FIG. 11 is a plot of tissue versus breath alcohol concentration acquired from the same clinical study as in FIG. 9.

FIG. 12 is a schematic illustration of an example embodiment of the present invention, combining a breath alcohol device and a tissue alcohol/analyte/biometric device.

FIG. 13 is a schematic illustration of an example embodiment of the present invention, combining a multivariate spectroscopic breath alcohol device and a tissue alcohol/analyte/biometric sensor with a shared light source and spectrometer.

FIG. 14 is a schematic illustration of an example embodiment of the present invention, combining a multivariate spectroscopic breath alcohol device and a tissue alcohol/analyte/biometric sensor with a shared spectrometer.

FIG. 15 is a schematic illustration of an example embodiment of the present invention, combining a multivariate spectroscopic breath alcohol device and a tissue alcohol/analyte/biometric sensor.

FIG. 16 is an illustration of plots of alcohol measurement results obtained from skin tissue obtained from a spectroscopic tissue alcohol device in accord with the present invention.

FIG. 17 is an illustration of biometric measurement results obtained from skin tissue obtained from a spectroscopic tissue biometric device in accord with the present invention.

### DETAILED DESCRIPTION OF THE INVENTION

These examples should not be construed as limiting to the invention as one skilled in the art recognizes that other embodiments exist that provide substantially the same function. For example, while the majority of the disclosure relates to near infrared spectroscopic measurements, Raman measurements (and therefore Raman spectrometers) can also be suitable for the present invention.

Definitions. The term “biometric” refers to a biological characteristic that can be used to identify or verify the identity of a specific person or subject. The term “attribute” refers to an analyte or a biometric. The present invention addresses the need for analyte measurements of samples utilizing spectroscopy where the term “sample” generally refers to biological tissue or breath. The term “subject” generally refers to a person from whom a sample measurement was acquired. The term “controlled environments” refers to any environment where the presence of an individual is subject to any restrictions related to alcohol, substances of abuse, or identity. This includes, but is not limited to, business offices, government buildings, probation centers, locations where individuals are located under home arrest, community corrections facilities, alcohol and substance of abuse treatment centers, public places incorporating check-in kiosks, vehicles, airplanes, buses, cars, trucks, trains, machinery, roadsides, streets, and facilities or equipment with restricted access such as nuclear power plants and weapons storage facilities.

Multivariate Spectroscopic Breath Alcohol Device

Breath alcohol devices can be classified into one of three general categories: electrochemical (fuel cell), semiconductor, or spectroscopic. Both electrochemical and semiconductor-based breath testers are inherently univariate in nature in that they measure a single current or voltage that is related to the concentration of alcohol. Both approaches are susceptible to chemical interferents that can generate their own electrical current or voltage. Furthermore, there is no straightforward method for adding the ability to discriminate between electrical signals due to alcohol and electrical signals due to other chemical species. As a result, spectroscopic based breath measurements (typically those based on infrared spectroscopy) are used in many evidential applications of breath alcohol measurements.

Many existing spectroscopic breath alcohol devices measure the absorbance of a breath sample at a single wavelength. The specific wavelength measured is chosen to coincide with a significant absorption band of ethyl alcohol. Other chemical species, such as acetone, can also absorb at the selected wavelength. Consequently, if these species are present in the breath sample, erroneous alcohol measurements can result. In order to address this risk, some breath devices incorporate measurements at additional wavelengths corresponding to the species of concern. If signal is detected at the added wavelengths, chemical interferents are suspected and the measurement is aborted.

The present invention discloses methods and apparatuses that can determine the concentration of the analyte of interest despite the presence of other chemical species thus obviating the need to abort the measurement. The present invention involves spectroscopic measurements with a plurality of wavelengths (referred to as multivariate spectroscopy) in order to accurately determine alcohol concentration when one or more interfering chemical, instrumental, or environmental interference is present in the breath sample

There are many multivariate spectroscopic methods known in the art that are relevant to quantitative determination of analyte concentrations or other attributes or properties. Some examples of multivariate spectroscopic methods that are suitable for the present invention include, but are not limited to, partial least squares regression (PLS), linear regression, multiple linear regression (MLR), classical least squares regression (CLS), neural networks, discriminant analysis, principal components analysis (PCA), principal components regression (PCR), cluster analysis, K-nearest neighbors, or combinations thereof. For demonstrative purposes, Classical Least Squares (CLS) and Partial Least Squares (PLS) will be discussed in more detail.

Classical Least Squares (CLS)

The Beer-Lambert law is commonly invoked in absorption spectroscopy to elucidate the relationship between the measured signal and the property of interest (alcohol concentration). For a sample containing a single absorbing analyte that is spectroscopically measured at a single wavelength, the Beer-Lambert Law can be expressed as:

A_{λ}=ε_{λ}lc (eq. 1)

where A_{λ} is the absorption of the sample at wavelength λ, ε_{λ} is the absorptivity of the single analyte in the sample at wavelength λ, l is the pathlength that the light travels through the sample, and c is the concentration of the analyte. As such, the Beer-Lambert Law states that a linear relationship between the absorbance of the sample and the concentration of the analyte in the sample. In order to determine the concentration of the analyte in practice, ε_{λ} and l must be known quantities such that upon experimental measurement of A_{λ}, the concentration (c) is the only remaining unknown.

The Beer-Lambert Law can be extended to samples containing more than one analyte; however, additional wavelengths must be measured in order to determine the property of interest. For example, a sample containing 2 analytes must be measured at two wavelengths according to the following equations:

A_{λ1}=ε_{αλ1}lc_{α}+ε_{βλ1}lc_{β} and A_{λ2}=ε_{αλ2}lc_{α}+ε_{βλ2}lc_{β} (eqs. 2 and 3)

where α and β represent the 2 analytes and 21 and 22 are the two measured wavelengths.

From a mathematical perspective, the number of unknowns (concentrations) in the system of equations can never exceed the number of equations, thus necessitating the measurement of additional wavelengths (to add more equations) and complete characterization of the sample (all ε terms must be separately determined and the pathlength must be known). It can be shown that multi-wavelength measurements based upon the Beer-Lambert law are a special case of Classical Least Squares (CLS) which is shown in equation 4.

A=KC (eq. 4)

Where K is a matrix containing the absorptivities of each analyte (one analyte per column of K) that have been multiplied by the pathlength, C is a matrix the concentrations of the analytes (one analyte per row), and A is the matrix of absorption spectra (each measurement is a column). In some applications of CLS the K matrix is experimentally determined by measuring each analyte independently of the others, thus obtaining a “pure component” spectrum of that analyte. Each pure component spectrum becomes a column of the K matrix. If necessary, the pure components are scaled to the proper pathlength (e.g. if the pure components were acquired using a different pathlength than what will be used to make future measurements). In other applications, pure component spectra may not be readily available (e.g. only mixtures of analytes are available). In this case, as long as a sufficient number of mixtures are available with differing and known analyte concentrations, equation 4 can be solved for K by acquiring a spectroscopic measurement of each mixture (each measurement is a column of the A matrix). As C is known for the mixtures, the only unknown in equation 4 is K, which can be determined via linear algebra.

Once K has been determined, the concentrations of all analytes in future measurements can be determined using equation 5 or 6.

Cₑₛₜ=A/K and Cₑₛₜ=AK⁻¹ (eqs. 5 and 6)

Where K⁻¹ is the inverse (or pseudo inverse) of K. The fact that CLS yields concentration estimates for all analytes, rather than for example alcohol alone, can be an advantage in some measurement scenarios.

CLS can be limited by the need to know all analytes that will be present in future measurements such that they are included in the K matrix. Furthermore, spectra must be acquired with at least as many wavelengths as there are analytes to be measured (with more wavelengths being desirable). If a new analyte were to be encountered or the constituents of a sample not fully characterized (e.g. if any analytes were absent from the K matrix), erroneous concentration estimates would result for all analytes since the K matrix would be invalid.

There are several strategies for accommodating new analytes including measurement of new pure components (and correspondingly adding new columns to the K matrix), or augmented CLS approaches such as PACLS, described by Haaland. Another consideration is that CLS can be sensitive to changes in spectrometer baseline and responsivity over time. In some cases, the methods described by Haaland can be useful in addressing these limitations as well.

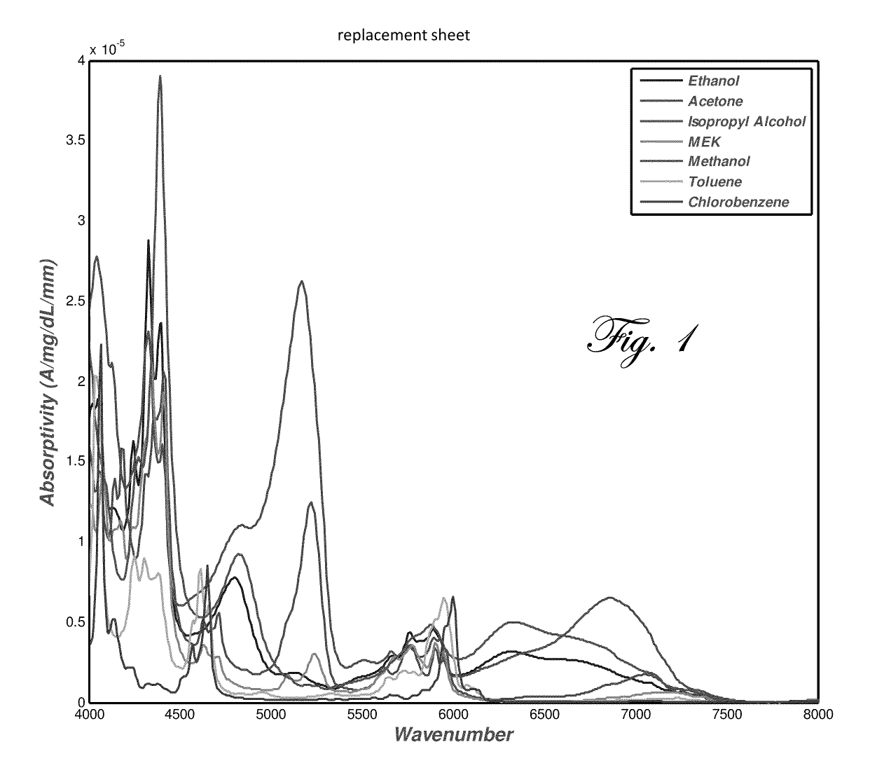

Advantages of CLS can be shown with a simulation of mixtures containing 7 analytes. Spectra of pure ethanol, isopropyl alcohol, methanol, acetone, toluene, methyl ethyl ketone (MEK), and chlorobenzene were obtained using a Fourier Transform Near-infrared (FT-NIR) spectrometer operating at 16 cm⁻¹ resolution. These spectra (called “pure components”) are shown in FIG. 1 and were used to form the K matrix (each pure component was a column of K) for the simulation. 1000 mixture spectra (A matrix) were then generated using the 7 components and a Latin-Hypercube design with a concentration range of 0 to 300 mg/dL for each component. This resulted in a 1000 row by 7 column C matrix where each row of C contained the concentrations of the analyte in the corresponding column of K. The squared correlations (r²) between components were less than 0.000001 for all analyte pairs.

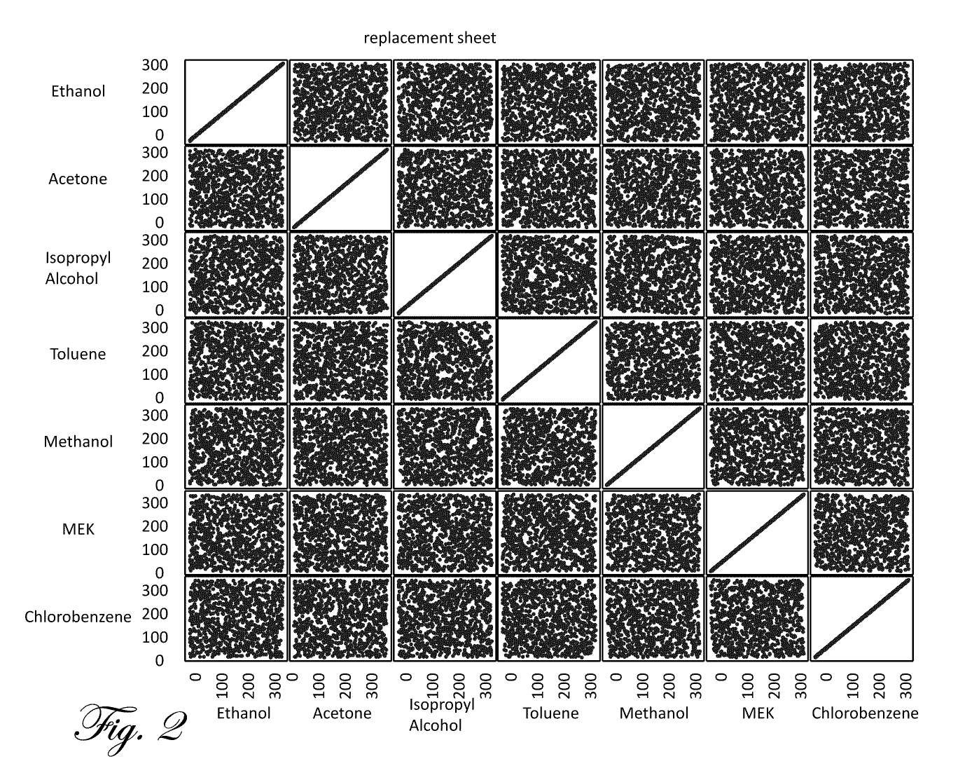

The simulated spectra (A matrix) and the K matrix were used with equation 7 to determine estimated concentrations, Cₑₛₜ. FIG. 2 shows the resulting concentration estimates (Cₑₛₜ) plotted against the known concentrations (C) of the analytes. Along the diagonal of FIG. 2 are the estimated concentrations of each analyte versus their known concentrations while the off diagonal charts are the estimated concentrations of each analyte versus the concentrations of the other analytes in the simulated spectra. The excellent agreement of the analyte concentration estimates relative to their known concentrations (the diagonal windows) combined with the absence of correlation with the concentrations of the other analytes in the mixtures (the off diagonal windows) indicates that each analyte can be measured independently of the other analytes present in the mixtures. Admittedly, this simulation is optimistic in the sense that no noise or spectrometer drift is present in the simulated spectra. However, the simulation does show that accurate concentrations can be obtained for all analytes simultaneously using multivariate methods such as CLS even when other analytes with overlapping spectroscopic features are present (see FIG. 1).

Inverse Multivariate Methods

Spectral measurements of complex media, such as human breath and tissue, can be comprised of many overlapping spectral signatures from a large number of chemical analytes. While feasible in some situations depending on the measurement objectives, the Beer-Lambert/CLS class of approaches can be difficult to implement due to the large number of potential variables and analytes. In such cases, alternative multivariate analysis methods can be used to decouple the signal of the analyte of interest from the signals of other analytes in the system (interferents). Partial Least Squares (PLS) regression is a inverse multivariate analysis method that can be applied to quantitative analysis of spectroscopic measurements and will be used for demonstrative purposes for the remainder of the disclosure. However, other inverse multivariate analysis methods such as Principal Components Regression (PCR), Ridge Regression, Multiple Linear Regression (MLR) and other methods such as Neural Networks are also suitable for use in the present invention. One skilled in the art will recognize that other methods of similar functionality can also be applicable.

Regardless of the specific algorithm employed, inverse multivariate methods attempt to find a solution for the regression coefficients, b, in equation 7.

y=Xb (eq. 7)

where y are the concentrations of the analyte of interest (for example ethanol), X is a matrix of spectral measurements, and b is the vector of regression coefficients. In words, the regression vector is a set of spectral weights (one per wavelength in the spectrum) that relates the spectral measurement to the property of interest (in this case ethanol concentration). The process of determining the regression coefficients is sometimes referred to as the calibration phase.

As an illustrative example of the calibration phase in PLS regression, a set of spectroscopic measurements is acquired (X) where each spectroscopic measurement has a corresponding known value (also called a reference value) for the property of interest (y; in this example blood alcohol concentration). The calibration spectral data are then decomposed into a series of factors (spectral shapes that are sometimes called loading vectors or latent variables) and scores (the magnitude of the projection of each spectrum onto a given factor) such that the squared covariance between the reference values and the scores on each successive PLS loading vector is maximized. The scores of the calibration spectra are then regressed onto the reference values in a multiple linear regression (MLR) step in order to calculate a set of spectral weights (one weight per wavenumber in the spectra) that minimizes the analyte measurement error of the calibration measurements in a least-squares sense. The spectral weights are called the regression vector (b). Once the calibration phase is completed, subsequent measurements of the property of interest are obtained by calculating the vector dot product of the regression vector and each measured spectrum.

An advantage of PLS and similar methods is that the ε terms in the Beer-Lambert Law (and thus the complete composition of the sample) do not need to be known. Furthermore, inverse methods tend to be more robust at dealing with nonlinearities in the spectral measurement and spectroscopic signals caused by instrumental drift, light scattering, environmental noise, and chemical interactions.

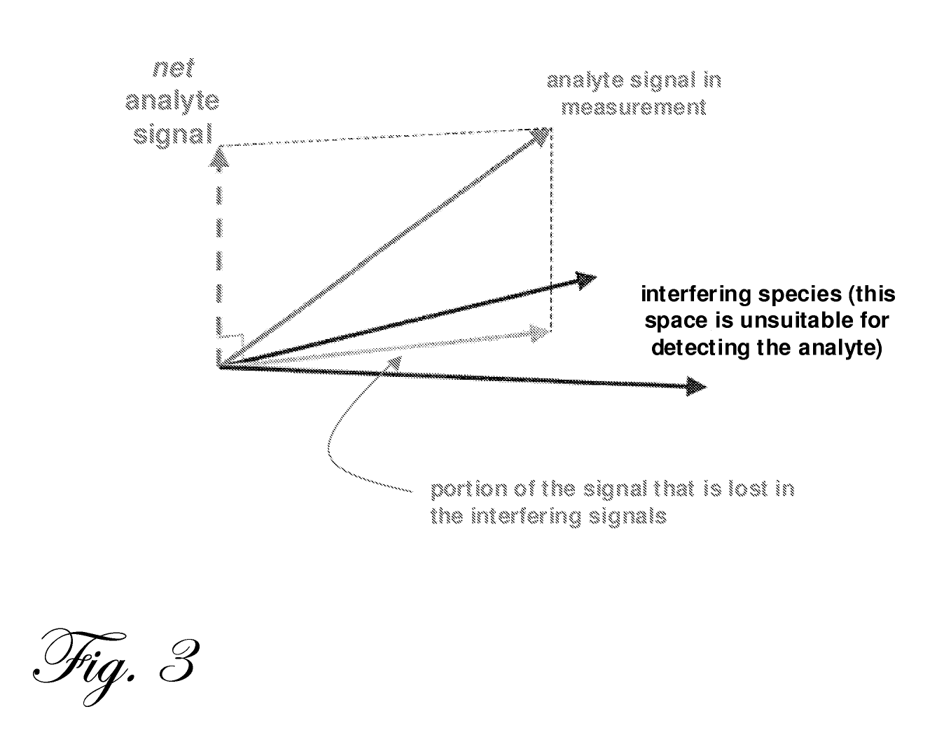

Functionally, the multivariate calibration (PLS or otherwise) in the present invention provides an ability to determine the part of the spectroscopic signal of alcohol that is effectively orthogonal (contravariant) to the spectra of all interferents in the sample. This part of the signal is referred to as the net attribute signal and can be calculated using the regression vector (b) described above using equation 9. If there are no interfering species, the net attribute spectrum is equal to the pure spectrum of alcohol. If interfering species with similar spectra to the analyte are present, the net attribute signal will be reduced relative to the entire spectrum. The concept of net attribute signal for a three-analyte system is depicted graphically in FIG. 3.

$$NAS = \frac{\hat{b}}{{‖ \hat{b} ‖}_{2}^{2}} \qquad ( eq . 8 )$$

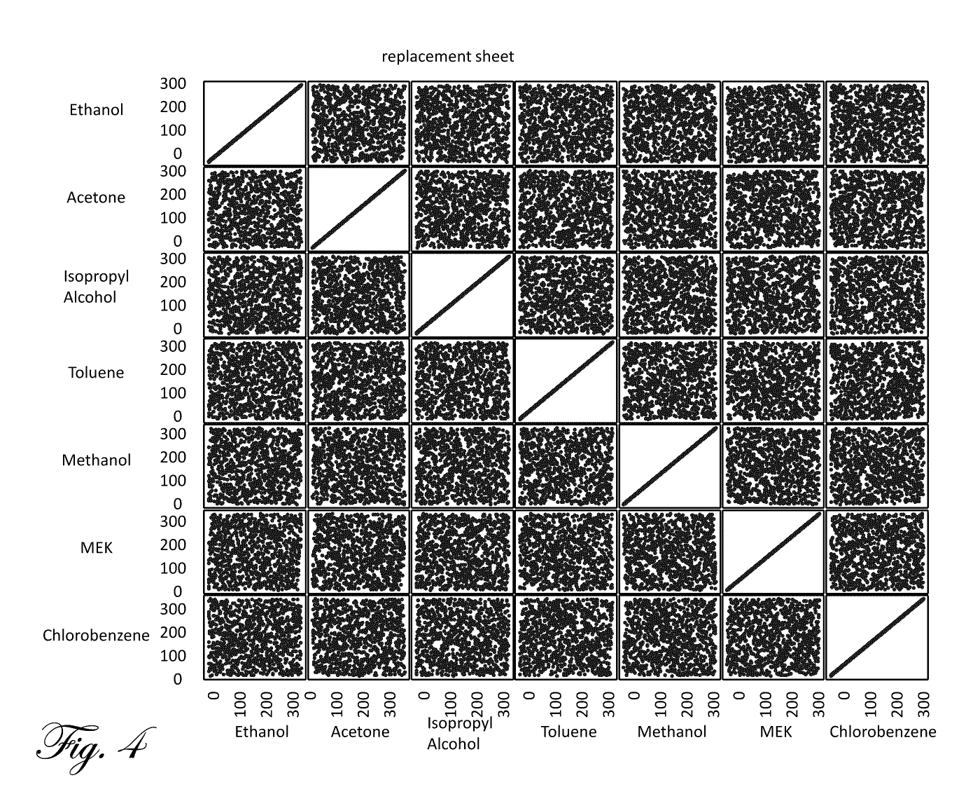

FIG. 4 shows the results of PLS regression on the same simulated measurements described in the CLS section. In the PLS case, a regression vector (b) was generated for each analyte. Furthermore, regression coefficients can be obtained, but it is not required, for multiple analytes. In such cases, one would have a b vector for each analyte whose concentration is desired. It is important to note that there is no need to obtain regression vectors for all analytes if a single analyte (ethanol) or subset of analytes is of interest. It is recognized that a PLS model for each analyte present in a mixture can outperform the CLS case where a single step (using the K matrix or its inverse) is used to estimate all analyte concentrations simultaneously. This is because inverse methods are inherently less sensitive to the presence of unknown analytes as well as instrument drift or variation.

Multivariate Evaluation of Measurement Risk

Multivariate methods, whether direct or inverse, have additional advantages relative to current breath alcohol measurements based on spectroscopy. In particular, multivariate methods offer metrics that enable a prospective measurement to be assessed for quality or risk. Measurements with an associated “high” risk can be deemed outliers and no measurement result reported. These types of metrics can be of particular importance in detecting attempts to circumvent or spoof the measurement or when the instrument is not operating properly.

The spectral residual magnitude is an example metric that determines the magnitude of the portion of a prospective measurement that is not explained by the model (e.g. the portion of the spectrum not explained by the K matrix in the CLS case) and compares that magnitude to those of normal measurements. If the prospective measurement exhibits a higher than normal residual magnitude, there is an increased probability that there are unexpected spectral shapes present. The measurement can then be disqualified rather than report a suspect analyte concentration. Other metrics, such as the Mahalanobis distance, offer similar information that can help enable outlier or suspect measurements to be identified. Furthermore, some multivariate metrics such as those disclosed by Maynard et. al. in 20040204868, “Reduction of errors in non-invasive tissue sampling”, incorporated herein by reference, can provide feedback to the user or test administrator regarding potential causes of the higher than normal risk as well as potential remedies.

Apparatuses for Acquiring Multi-Wavelength Absorbance Spectra

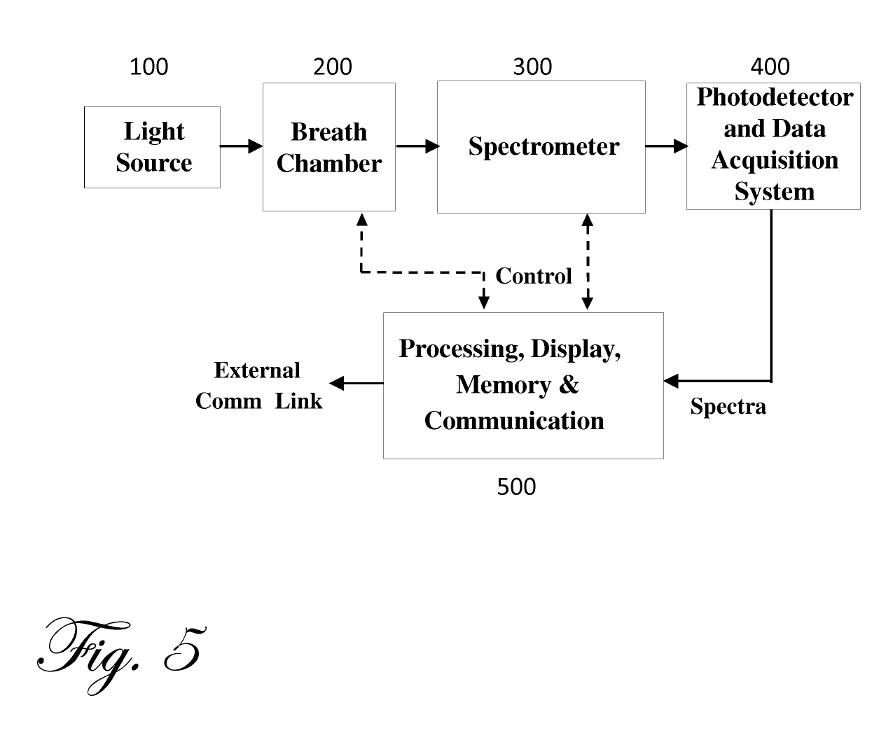

In order to perform multivariate breath alcohol measurements, an apparatus that enables spectroscopic measurements at multiple wavelengths can be used. FIG. 5 shows a diagram of an apparatus comprising 5 subsystems that is suitable for making multivariate spectroscopic breath measurements. The light source (100) generates light at the desired wavelengths to be measured. Suitable embodiments of the light source (100) are filament lamps such as quartz tungsten halogen (QTH) lamps, black body emitters (e.g. resistive elements such as igniters), or solid state light source such as light emitting diodes, gas lasers (e.g. Helium Neon), VCSEL's, or other semiconductor based light sources or lasers.

In FIG. 5 the light emitted by the light source (100) is directed to the breath chamber (200) where the light interacts with the sample (e.g. human breath or a calibration standard). This interaction can be in transmission where the light passes through the sample once or multiple times using mirrors. The breath chamber (200) can also be designed such that the breath of the person being tested flows through the chamber. Suitable embodiments of the breath chamber (200) are known in the art such as those found in existing electrochemical, semiconductor, and spectroscopic based breath testing devices.

The light from the breath chamber (200) is then directed to the spectrometer subsystem (300). The spectrometer subsystem can resolve or separate different wavelengths of light from each other. Two general approaches to spectrometer subsystem (300) design that are equally suitable for the purposes present invention are described below. For the purposes of this invention the term “dispersive spectrometer” indicates a spectrometer based upon any device, component, or group of components that spatially separate one or more wavelengths of light from other wavelengths. Examples include, but are not limited to, spectrometers that use one or more diffraction gratings, prisms, or holographic gratings. For the purposes of this invention the term “interferometric/modulating spectrometer” indicates a class of spectrometers based upon any device, component, or group of components that either modulate different wavelengths of light to different frequencies in time or selectively transmits or reflects certain wavelengths of light based upon the properties of light interference or through modulation devices such as choppers or filter wheels. Examples include, but are not limited to, Fourier transform interferometers, Hadamard spectrometers, Sagnac interferometers, mock interferometers, Michelson interferometers, one or more etalons, acousto-optical tunable filters (AOTF's), mechanical or optical choppers, filter wheels, and one or more solid state light sources that are scanned or modulated.

The terms “solid state light source” or “semiconductor light source” refer to all sources of light, whether spectrally narrow (e.g., a laser) or broad (e.g., an LED) that are based upon semiconductors which include, but are not limited to, light emitting diodes (LED's), vertical cavity surface emitting lasers (VCSEL's), horizontal cavity surface emitting lasers (HCSEL's), quantum cascade lasers, quantum dot lasers, diode lasers, or other semiconductor lasers. Furthermore, plasma light sources and organic LED's, while not strictly based on semiconductors, are also contemplated in the embodiments of the present invention and are thus included under the solid state light source and semiconductor light source definitions for the purposes of this disclosure.

One skilled in the art recognizes that spectrometers based on combinations of dispersive and interferometric/modulating properties, such as those based on lamellar gratings, are also suitable for the present invention. Several types of spectroscopic “signals” are applicable to the present invention. Signals can comprise any measurement obtained concerning the spectroscopic measurement of a sample or change in a sample, e.g., absorbance, reflectance, intensity of light returned, fluorescence, transmission, Raman spectra, or various combinations of signals, at one or more wavelengths.

The light exiting the spectrometer subsystem (300) is then directed to a photodetector and associated data acquisition subsystem (400). The photodetector and data acquisition subsystem (400) converts the resolved wavelengths of light into electrical signals and then to a digitized representation of the electrical signals. Some examples of suitable photodetectors are single or multi-element devices comprised of InGaAs, InAs, Ge, PbSe, PtSi, PbS, InSb, or silicon based detectors such as CCD's or CID's. The remainder of the photodetector and data acquisition subsystem (400) amplifies and filters the electrical signal from the detector and then converts the resulting analog electrical signal to its digital representation with an analog to digital converter. Additional steps such as digital filtering and re-sampling of the digital signal can also be performed in some embodiments.

The processing, display, memory, and communications subsystem (500) performs multiple functions such mathematical transforms that are applied to the digital signal obtained from the photodetector and data acquisition subsystem (400), performing signal outlier checks to ensure the measured signal is appropriate, signal preprocessing in preparation for determination of the alcohol concentration or other attribute of interest, determination of the alcohol concentration or other attribute of interest, system status checks, all display and processing requirements associated with the user interface, and data transfer and storage. In some embodiments, the computing subsystem is contained in a personal computer or laptop computer that is connected to the other subsystems of the invention. In other embodiments, the computing subsystem is a dedicated, embedded computer. The results can be reported visually on a display, by audio and/or by printed means. Additionally, the results can be stored to form a historical record of the attribute. In some embodiments, the results can be stored and transferred to a remote monitoring or storage facility via the internet, phone line, or cell phone service.

The processing, display, memory, and communications subsystem (500) includes a central processing unit (CPU), memory, storage, a display and preferably a communication link. An example of a CPU is the Intel Pentium microprocessor family. The memory can be, e.g., static random access memory (RAM) and/or dynamic random access memory. The storage can be accomplished with non-volatile RAM or a disk drive. Suitable embodiments for the display include liquid crystal displays (LCD's), LED's, CRT's, plasma displays, or any other color or black and white display. The communication link can be, as examples, a high speed serial link, an Ethernet link, or a wireless communication link.

The processing, display, memory, and communications subsystem (500) can also contain a communication link that allows transfer of a subject's alcohol measurement records and the corresponding spectra to an external database. In addition, the communication link can be used to download new software to the computer and update the multivariate calibration model. The computer system can be viewed as an information appliance. Examples of information appliances include personal digital assistants, web-enabled cellular phones and handheld computers.

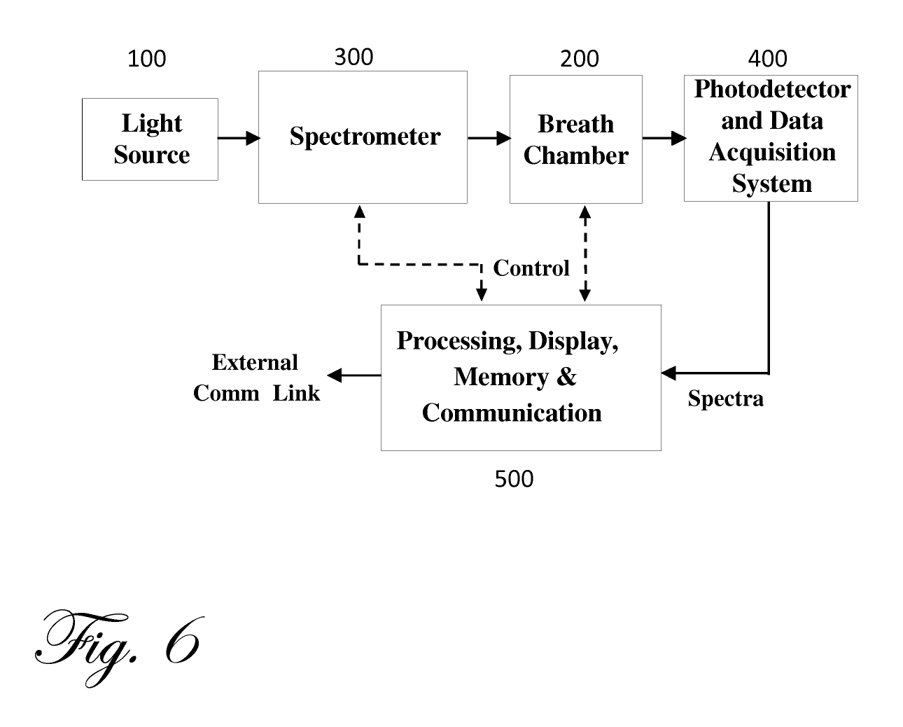

FIG. 6 shows an alternative arrangement of the subsystems shown in FIG. 5 where the physical locations of the breath chamber (200) and the spectrometer subsystem (300) have been transposed. For some embodiments, such as those incorporating Michelson or similar interferometers, the arrangement in FIG. 6 can offer performance advantages relative to the arrangement shown in FIG. 5. One skilled in the art recognizes the different types of spectrometer subsystems (300) available and in combination with the optical design of the system can determine the arrangement (light source, breath chamber, spectrometer or light source, spectrometer, breath chamber) that is preferable.

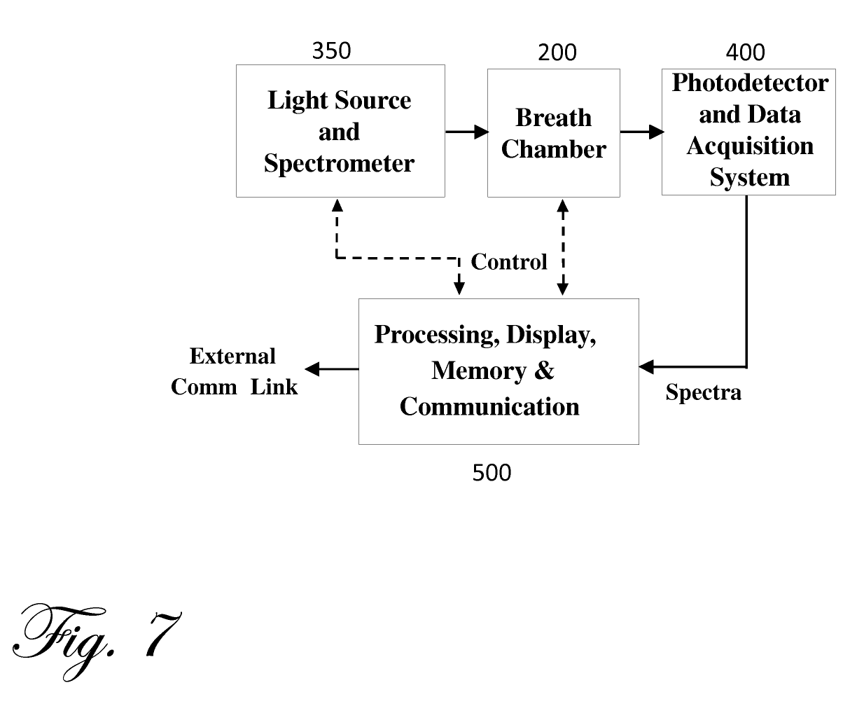

FIG. 7 shows a variant of FIG. 6 where the light source and spectrometer subsystems (100 and 300) are combined into a single subsystem (350). An example of an embodiment of the combined light source and spectrometer subsystem (350) is comprised of multiple solid state light sources, such as VCSEL's that emit at different wavelengths. These light sources are each modulated at different frequencies either by cycling their power states or through optical or mechanical choppers. The result is that each wavelength of light to be measured has a different frequency such that a single element detector can simultaneously measure all wavelengths. Additional suitable embodiments of this type of light source/spectrometer combination are described in U.S. provisional application 61/147,107, filed Jan. 25, 2009, which is incorporated herein by reference.

Combination of Breath Alcohol Device with Multivariate Tissue Alcohol Device

Breath devices are limited by several concerns regarding falsely elevated alcohol measurements. A waiting period is typically observed prior to performing a breath alcohol measurement in order to ensure that mouth alcohol is not present as it is much higher in concentration than alcohol expired from the lungs and therefore does not adequately reflect the blood alcohol concentration. The waiting period is typically 10-20 minutes and requires direct observation, e.g., by a law enforcement official. Any burping or vomiting can indicate stomach alcohol being introduced to the mouth, which resets the waiting period. The waiting period is a significant issue for breath testing as it prevents the observer from performing other duties.

Multivariate tissue alcohol measurements can be used in conjunction with breath measurements and remove the requirement for a waiting period in all cases. As tissue alcohol measurements determine the alcohol concentration in skin tissue, mouth and stomach alcohol are not of concern: they do not contribute to the tissue alcohol measurement. Consequently, both tissue and breath alcohol measurements can be performed immediately, without any waiting period. If both the breath and tissue alcohol measurements are below the legal limit, mouth and stomach alcohol are not of concern as the person is not under the legal limit. If both the breath and tissue alcohol are above the legal limit, mouth and stomach alcohol cannot be significant contributors to the breath measurement as they would not influence the tissue alcohol measurement. In other words, the breath alcohol result is more trustworthy, even without the waiting period, as mouth and stomach alcohol have been ruled out. In cases where the breath alcohol measurement is above the legal limit and the tissue alcohol measurement is below, mouth and stomach alcohol can be a plausible explanation of the difference. In this case, a waiting period can be instituted and a 2^{nd} breath test administered. Alternatively, the tissue alcohol measurement can be used in lieu of the breath measurement in some applications. Thus, the combination of breath and tissue alcohol measurements can obviate the need for a waiting period in the majority of testing cases.

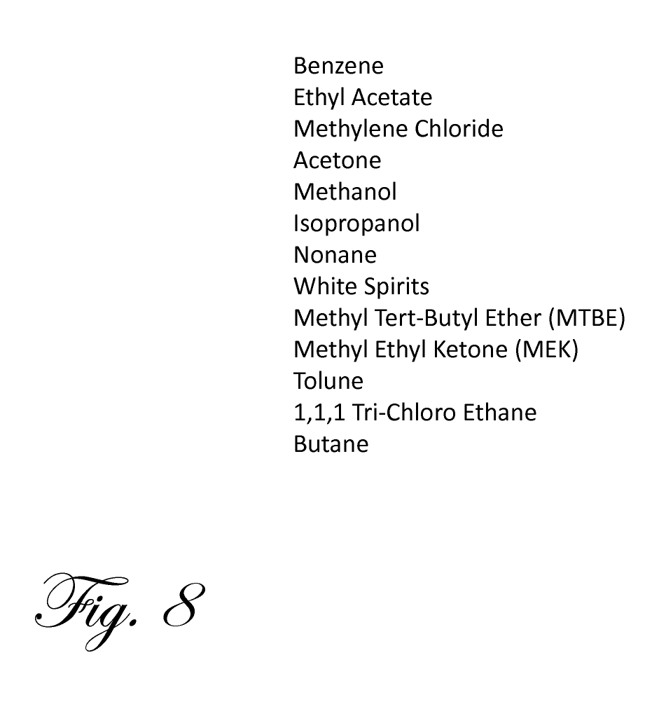

Another concern for breath alcohol measurements is the potential presence of chemical interferents in the breath sample. Whether fuel cell (electrochemical), semiconductor, or spectroscopic-based, there is the potential for other substances to erroneously contribute to the alcohol measurement. FIG. 8 shows a list of exemplary breath interferents that are known in the art. These interferents can be expelled in the breath of the person being tested or present in the ambient air (e.g. from automobile emissions). The interferent is generally in the vapor phase and can contribute to the alcohol measurement if present.

A multivariate tissue alcohol sensor, however, does not measure analytes in the vapor phase. Instead, the concentration of liquid ethanol within the skin is measured. Furthermore, the tissue sensor can be in physical contact with the skin, which precludes airborne chemicals from contributing to the measurement. Consequently, similar to the scenarios described for mouth alcohol, the combination of the breath and tissue alcohol measurements provides supplemental information that reduces or eliminates the concerns regarding chemical interferences. For example, positive results on both breath and tissue measurements indicate that interferences are unlikely as it is extremely unlikely that a breath interferent that falsely elevates its result will be combined with a tissue interferent on the skin that falsely elevates its result in a similar manner. Environmental noises, such as RF interference can also be expected to influence breath and tissue alcohol sensors and reduction of sensitivity to those are also considered as part of the advantages imparted by the present invention.

During prosecution, breath measurements can also suffer from arguments related to the blood breath ratio (BBR) which is a conversion between the concentration of alcohol in the air and the concentration of alcohol in the blood. This conversion varies between people and conditions due to physiology and environmental variables such as temperature. Extensive clinical testing is required to determine a person's BBR, thus it is not known at the time of alcohol measurements performed in law enforcement. Consequently, a BBR of 2100 is applied to all tests within the United States. The 2100 BBR is lower than the true value for most people, which gives the benefit of the doubt to the defendant. However, there are individuals with BBR's lower than 2100 which results in overestimation of the blood alcohol concentration for these individuals. Defense attorneys routinely argue that their clients have BBR's lower than 2100 in order to create reasonable doubt. Incorporation of a multivariate tissue alcohol measurement can obviate this strategy as the BBR is inapplicable for tissue alcohol measurements. Thus, if both breath and tissue alcohol concentrations are above the legal limit, the BBR is no longer a sufficient argument for a person's innocence.

Another advantage of the combination of breath and tissue alcohol measurements is that unsupervised screening with the tissue alcohol measurement can be performed and positive measurements confirmed by a supervised breath alcohol test. Mouth and stomach alcohol are not of concern for the tissue alcohol screening test; only positive (above limit) tissue measurements are confirmed by a breath test. The breath and tissue alcohol devices can by physically independent from each other or incorporated into a single product or package. Furthermore, while the above scenarios typically describe methods for a tissue alcohol measurement to obviate the weaknesses of breath measurements, it is recognized that other approaches are possible. In some scenarios the breath-tissue combination can be used to provide additional protections to the person being tested. For example, if either the breath or the tissue alcohol measurement were below the legal limit the person would not be guilty of driving under the influence.

Another aspect of the present invention is the ability to incorporate the measurement of analytes other than alcohol into the measurement system. For example, spectroscopic methods, such as those described by Miller et. al. in “Minimally invasive spectroscopic system for intraocular drug detection”, Journal of Biomedical Optics 7(1), 27-33, incorporated herein by reference, have been applied to the detection and quantification of substances of abuse. As such the noninvasive spectroscopic measurement described in Ridder will contain the spectroscopic signals of substances of abuse if present within the measured tissue.

For the purposes of this invention, the term “analyte concentration” generally refers to the concentration of an analyte, such as alcohol. The term “analyte property” includes analyte concentration and other properties, such as the presence or absence of the analyte or the direction or rate of change of the analyte concentration, which can be measured in conjunction with or instead of the analyte concentration. While the term “analyte” generally refers to alcohol, other chemicals, particularly substances of abuse and alcohol byproducts, can also be determined with the present invention. The term “alcohol” is used as an example analyte of interest; the term is intended to include ethanol, methanol, ethyl glycol or any other chemical commonly referred to as alcohol. For the purposes of this invention, the term “alcohol byproducts” includes the adducts and byproducts of the metabolism of alcohol by the body including, but not limited to, acetone, acetaldehyde, and acetic acid. The term “alcohol biomarkers” includes, but is not limited to, Gamma Glutamyl Transferase (GGT), Aspartate Amino Transferase (AST), Alanine Amino Transferase (ALT), Mean Corpuscular Volume (MCV), Carbohydrate-Deficient Transferrin (CDT), Ethyl Glucuronide (EtG), Ethyl Sulfate (EtS), and Phosphatidyl Ethanol (PEth). The term “substances of abuse” refers to, but is not limited to, THC (Tetrahydrocannabinol or marijuana), cocaine, M-AMP (methamphetamine), OPI (morphine and heroin), OxyContin, Oxycodone, and PCP (phencyclidine).

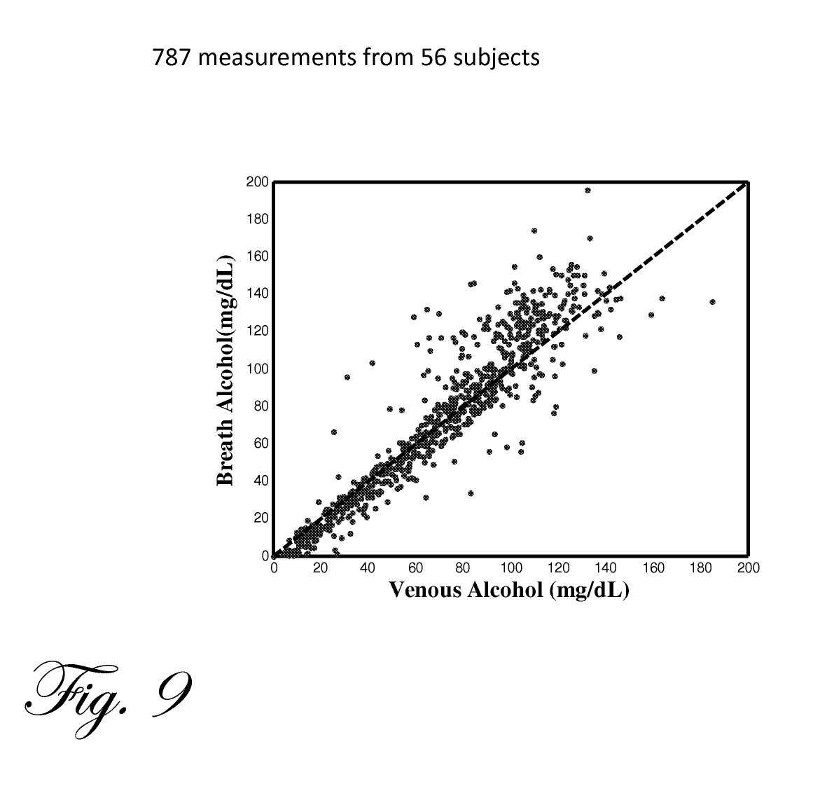

FIG. 9 shows 787 breath alcohol measurements versus contemporaneously measured venous blood alcohol concentration that were obtained from 56 subjects in controlled dosing study. As venous alcohol concentration represents the gold standard in the measurement of alcohol in people, the breath measurements would ideally fall on the dotted line in FIG. 9. However, FIG. 9 shows several breath measurements that are significantly higher or lower than their venous blood counterparts. These deviations are due in part to alcohol pharmacokinetics (the distribution of alcohol throughout the body), mouth alcohol events, potential presence of an interferents, and/or instrument error. An advantage of some embodiments of the present invention is that the combination of a tissue alcohol measurement with the breath measurement allows some of these erroneous measurements to be detected as they happen, without the need for a venous blood sample to be acquired.

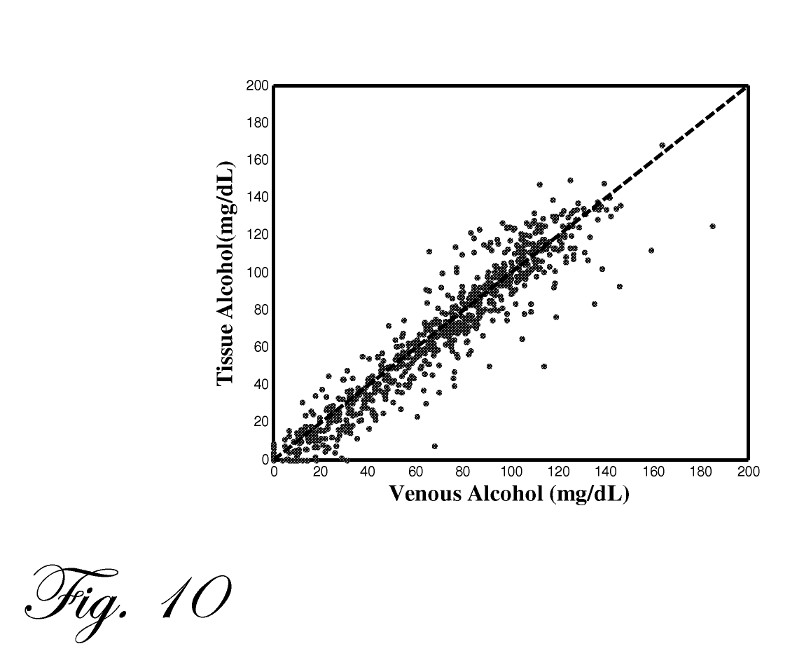

FIG. 10 shows contemporaneously measured tissue alcohol measurements plotted versus the same 787 venous blood alcohol measurements. Similar to FIG. 9, the measurements do not lie perfectly on the dotted line. However, as mouth alcohol is not an issue for the tissue measurements, the differences between the tissue and venous alcohol measurements are confined to pharmacokinetics, potential interference, and instrument error. Furthermore, the differences between tissue and venous alcohol (FIG. 10) are distinctly different than those observed for breath relative to venous (FIG. 9) which indicates that the breath and tissue measurements each contain unique information that can be used to improve overall measurement agreement with venous alcohol.

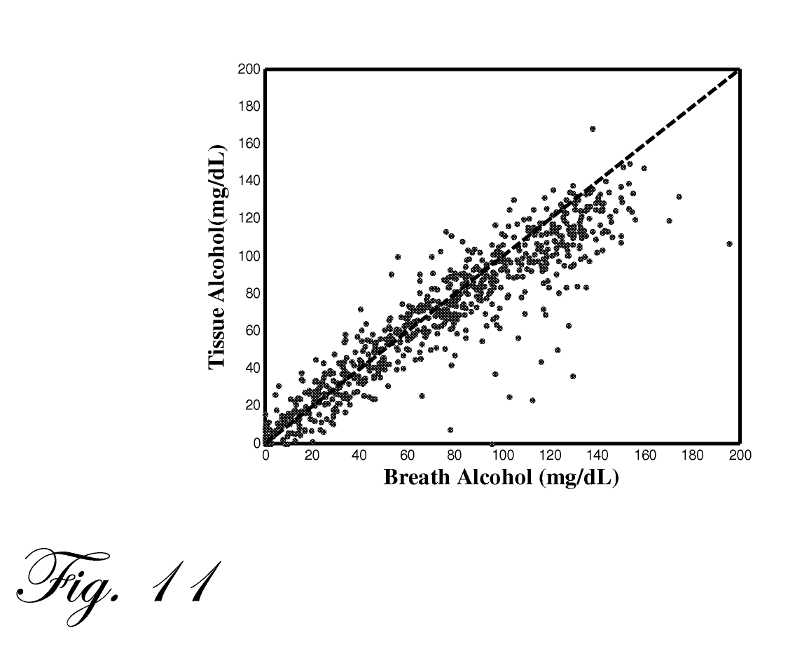

FIG. 11 shows the tissue alcohol measurements plotted versus the breath alcohol measurements. Several breath measurements exhibit alcohol concentrations above 60 mg/dL while the corresponding tissue alcohol concentrations are significantly lower. These differences could be due to the presence of mouth alcohol, interference, or due to alcohol pharmacokinetics (i.e. alcohol has not uniformly distributed in the body). Regardless of the cause, large difference between the tissue and breath alcohol concentrations provide valuable information that there is increased risk of poor agreement with venous. Depending on the situation, the test administrator can perform various corrective actions. For example, the administrator can choose to wait 10-20 minutes and repeat the test (to determine if mouth alcohol or pharmacokinetics was causing the difference), elect to acquire a blood sample based on the information imparted from the tissue and breath samples, or move locations and repeat the tests if a breath interferents is suspected to be present in the air. One skilled in the art recognizes other potential corrective actions that can be performed based on the information provided by the combined breath and tissue alcohol results.

Via a similar argument, when the breath and tissue alcohol results exhibit good agreement there is increased confidence that neither measurement is being significantly corrupted by pharmacokinetics, mouth alcohol, or interference. As such, the combination of tissue and breath alcohol assays constitutes greater proof of intoxication (or lack thereof) than either assay could individually provide. This greatly reduces avenues for defense attorneys to attack the accuracy of the alcohol results.

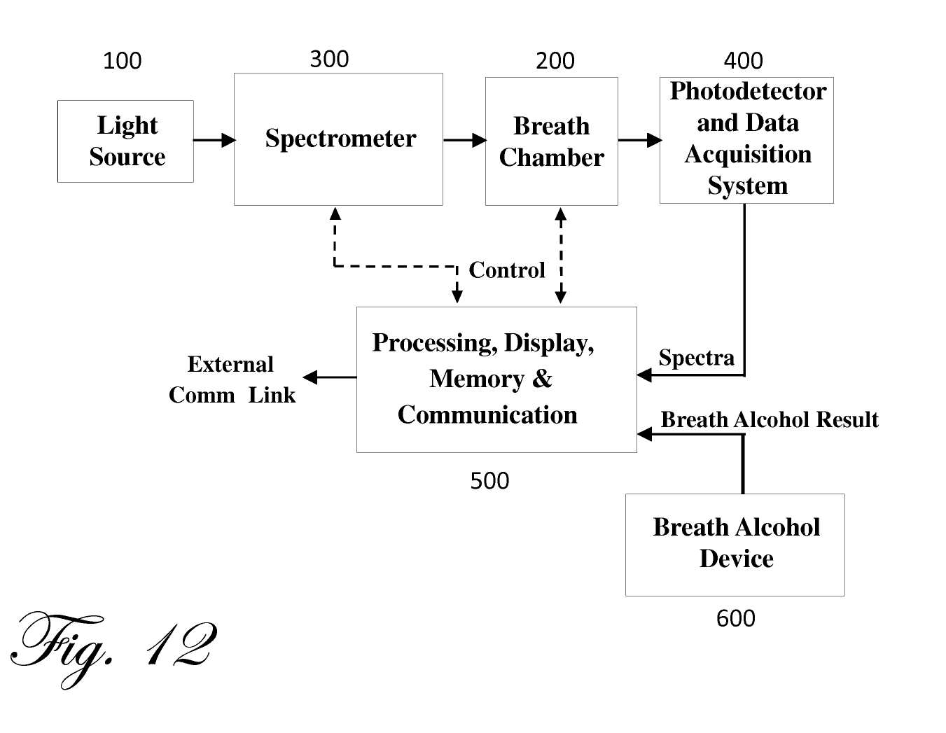

FIG. 12 shows a schematic of an embodiment that combines a breath alcohol device with a tissue alcohol device. In this embodiment the tissue alcohol device is comprised of multiple subsystems (100, 200, 300, 400, and 500) and the breath alcohol device is an additional subsystem (600) that communicates with the Processing, Display, Memory, and Communication subsystem (500). Thus, in some embodiments the breath device can be an independent spectroscopic, semiconductor, or electrochemical breath device that can be incorporated into the same physical package with the tissue alcohol device or be provided in a separate physical package. Furthermore, the breath device (600) can be removable and be “docked” with the tissue alcohol device (i.e. like a cordless phone) for charging and/or communication of results to the Processing, Display, Memory, and Communication subsystem (500).

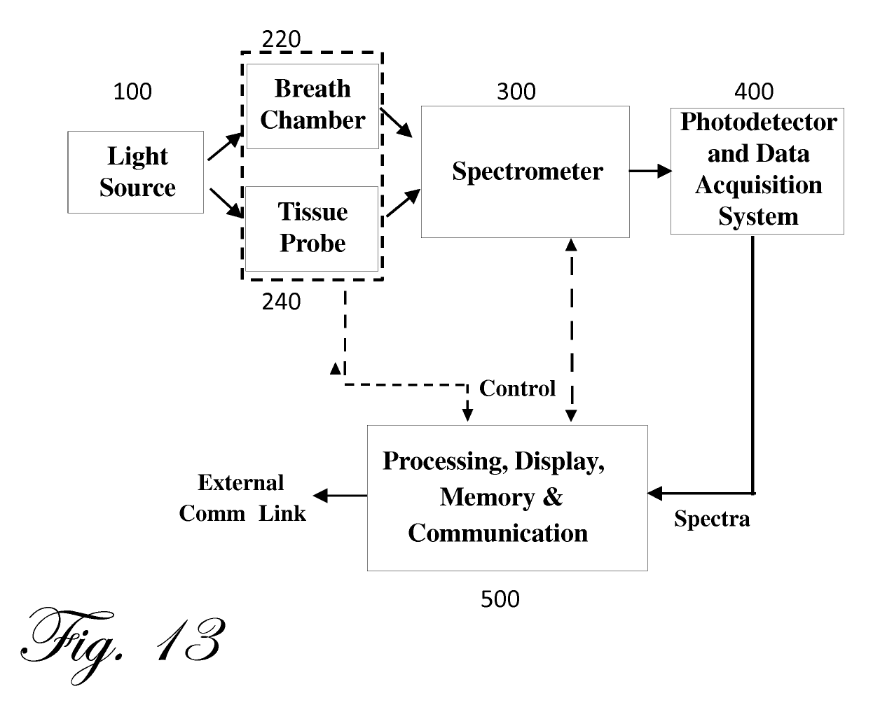

FIGS. 13-15 show schematics of embodiments that combine multivariate spectroscopic breath devices of the present invention with tissue alcohol devices. FIG. 13 shows an embodiment where the breath and tissue devices share a common Light Source subsystem (100), Spectrometer subsystem (300), Photodetector and Data Acquisition subsystem (400), and Processing, Display, Memory, and Communication subsystem (500). The embodiment has 2 subsystems for introducing a sample: one for breath samples (220) and one for tissue samples (240). One skilled in the art recognizes that additional sample introduction subsystems can be incorporated (e.g., if multiple tissue sites were to be measured). Furthermore, the present invention contemplates multiple approaches to measuring the breath and tissue in the embodiment shown in FIG. 13.

In some embodiments of the schematic shown in FIG. 13, the device can switch between the breath and tissue measurements such that only one is being performed at a given time. In other embodiments, the light to either or both of the breath and tissue measurements can be modulated such that both can be measured simultaneously. The signals from breath and tissue measurements would be decoupled in the Photodetector and Data Acquisition Subsystem (400). In other embodiments, the wavelengths of light of interest to breath measurements are different than those of interest to tissue measurements. Consequently, both can be measured simultaneously and the various wavelengths of interest for the breath and tissue measurements can be separated by the Photodetector and Data Acquisition Subsystem (400). Optical filtering after the Light Source subsystem (100) and prior to the spectrometer subsystem (300) can also be used to restrict the range of light wavelengths that contribute to the breath and tissue measurements.

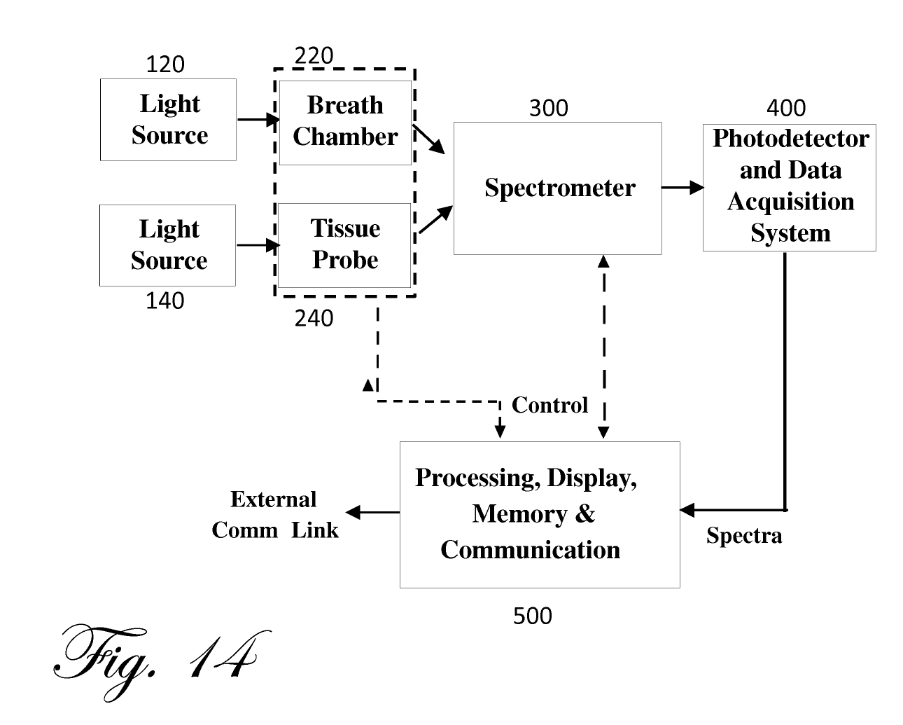

FIG. 14 shows another example embodiment of the present invention where the breath and tissue alcohol devices have dedicated Light Source subsystems (120 and 140) while sharing common a Spectrometer subsystem (300), Photodetector and Data Acquisition subsystem (400), and Processing, Display, Memory, and Communication subsystem (500). This can be advantageous in cases where the wavelengths of interest are significantly different for the breath and tissue cases, or in cases where one measurement uses a different type of light source. For example, the tissue alcohol Light Source (140) can incorporate a black body radiator and the breath alcohol Light Source (120) can incorporate a laser as a light source. One skilled in the art recognizes the large number of potential variants of the embodiment shown in FIG. 14. Similar to the embodiment of FIG. 13, the alcohol and breath measurements can be obtained serially via an optical, mechanical, or electronic switching mechanism or measured simultaneously and decoupled via the methods previously described.

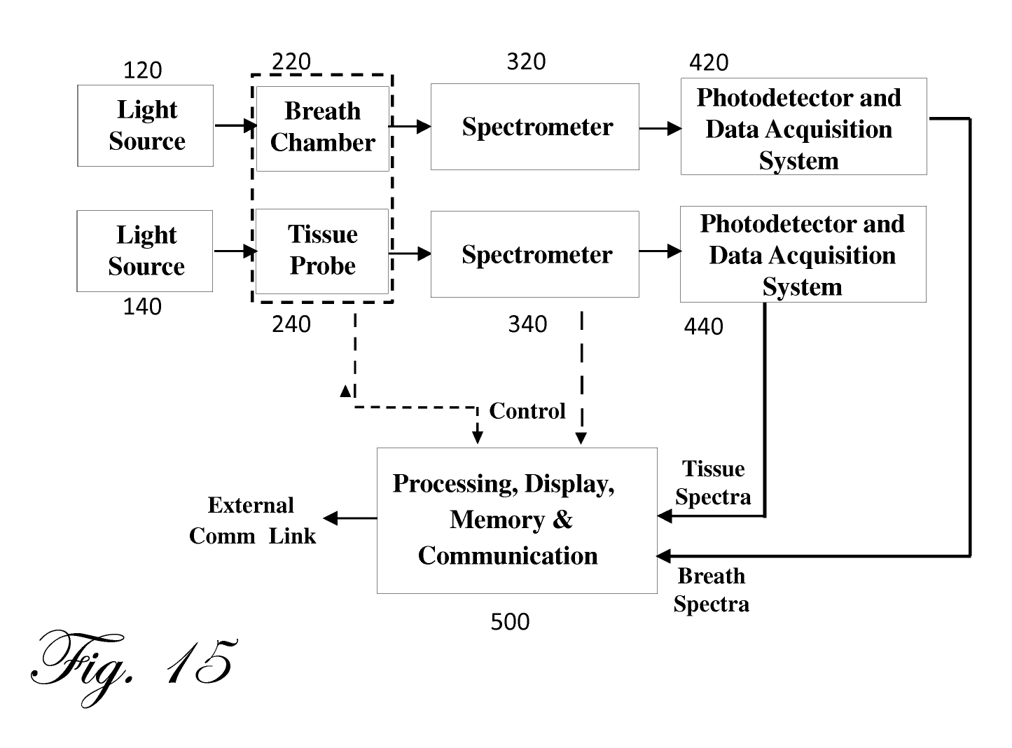

FIG. 15 shows another example embodiment of the present invention where the breath and tissue alcohol devices have dedicated Light Source (120 and 140) and spectrometer (320 and 340), and Photodetector and Data Acquisition (420 and 440) subsystems, with a common Processing, Display, Memory, and Communication subsystem (500). This can be advantageous in cases where the modalities of the alcohol and breath measurements are significantly different. For example, the tissue alcohol measurement can be based upon Raman spectroscopy and the breath measurement based upon infrared (IR) absorption. One skilled in the art recognizes the large number of potential variants of the embodiment shown in FIG. 15. Similar to the embodiments of FIGS. 13 and 14, the alcohol and breath measurements can be obtained serially via an optical, mechanical, or electronic switching mechanism or measured simultaneously and decoupled via the methods previously described.

Apparatuses Suitable for Tissue Alcohol and Analyte Measurements

Suitable spectroscopic systems for measuring alcohol and other analyte measurements in tissue are known in the art. In U.S. Pat. No. 7,403,804, titled “Noninvasive determination of alcohol in tissue,” incorporated herein by reference, Ridder et al. disclose a method for the noninvasive measurement of alcohol based on spectroscopic techniques that provides an alternative to the current blood, breath, urine, saliva, and transdermal methods. The device generally assumes passive contact between the noninvasive device and a tissue surface such as a finger, forearm, palm, or earlobe in order to measure the alcohol concentration in the tissue.

Additional apparatuses suitable for use in the present invention can be found in U.S. patent application Ser. Nos. 11/515,565 and 12/562,050, both titled “Apparatus and method for noninvasively monitoring for the presence of alcohol or substances of abuse in controlled environments,” incorporated herein by reference, in which Ridder et al. disclose apparatuses for the measurement of alcohol in tissue in a variety of controlled environments.

Additional apparatuses suitable for use in the present invention can be found in U.S. patent application Ser. No. 12/107,764, titled “Apparatuses for Noninvasive Determination of in vivo Alcohol Concentration using Raman Spectroscopy,” incorporated herein by reference, in which Ridder et al. disclose apparatuses for measuring alcohol in tissue using Raman spectroscopy.

Additional apparatuses suitable for use in the present invention can be found in U.S. patent application Ser. No. 11/393,341, titled “Apparatus and method for controlling operation of vehicles or machinery by intoxicated or impaired individuals,” incorporated herein by reference, in which Ridder et al. disclose apparatuses for measuring alcohol in order to prevent impaired operation of vehicles or machinery.

Additional apparatuses suitable for use in the present invention can be found in U.S. Patent Application No. 61/147,107, titled “System for Noninvasive Determination of Alcohol in Tissue,” incorporated herein by reference, in which Ridder et al. disclose embodiments of tissue alcohol measurement devices based on solid state and semiconductor based spectrometers. Additional apparatuses that can be used, or modified to be used, in the present invention are described in the following U.S. patents and applications, each of which is incorporated herein by reference: U.S. Pat. Nos. 7,606,608; 7,519,406; 7,509,153; 7,505,801; 7,333,843; 7,299,080; 7,233,816; 7,206,623; 7,183,102; 7,133,710; 7,038,774; 6,956,649; 6,864,978; 6,816,241; 6,640,117; 6,587,199; 6,587,196; 6,415,167; 6,040,578; 5,945,676; 5,747,806; 7,386,152; 7,347,365; 20060002598; 20090247840.

The above cited examples of tissue measurement apparatus are demonstrative and are not intended to be limiting. One skilled in the art recognizes that apparatuses derived in part from the above cited embodiments can also be suitable for the present invention.

Combination of Breath Alcohol Device with Multivariate Tissue Biometric Device

In community corrections, some individuals are assigned to home arrest such that they can continue to work and/or take care of their families. A frequent condition of home arrest is abstinence from alcohol. A challenge imposed by this condition is the need to verify compliance in a manner that isn't overly burdensome to law enforcement or other personnel. There are a few breath-based alcohol measurement approaches currently known in the art to serve this need. They generally involve the combination of a breath alcohol test with some means for verifying the identity of the person being tested. Voice recognition, face recognition, and remote video monitoring are used to perform the identity verification function.

The purpose of these approaches is to prevent a test administrator from physically needing to be present at the person's home in order to administer the test. However, concerns remain for these methods as the breath tester physically blocks a part of the face during the test which hampers face recognition and remote video monitoring techniques, while the mouth piece of the breath device makes speech, and thus voice recognition, difficult. An advantage of some embodiments of the present invention is that the combination of a breath alcohol device with a tissue biometric sensor eliminates these disadvantages since tissue sensor can be integral to the breath device such that the finger or part of the hand holding the device is used to perform the identity verification. Furthermore, the ergonomics of the device can be such that the tissue biometric sensor is located on the breath device in a manner that makes it difficult for the desired person to hold the device and perform the biometric test while another blows into the device.

Apparatuses Suitable for Tissue Biometric Measurements

In U.S. Pat. No. 6,628,809, titled “Apparatus and method for identification of individuals by near-infrared spectrum”, and in U.S. Pat. No. 6,560,352, titled “Apparatus and method of biometric identification or verification of individuals using optical spectroscopy”, each of which is incorporated herein by reference, Rowe et. al. disclose spectroscopic methods for determining the identity or verifying the identity of an individual using spectroscopic measurements of tissue. Such spectroscopic methods provide an alternative to existing fingerprint, voice recognition, video recognition, and bodily feature identification for the apparatuses contemplated with the present invention. Additional biometric systems that can be used, or modified to be used, in connection with the present invention are described in the following U.S. patents and published applications, each of which is incorporated herein by reference: U.S. Pat. Nos. 7,627,151; 7,620,212; 7,613,504; 7,545,963; 7,539,330; 7,508,965; 7,460,696; 7,394,919; 7,347,365; 7,263,213; 7,203,345; 7,147,153; 6,816,605; 6,560,352; 20090245591; 20090148005; 20090092290; 20090080709; 20090074255; 20090046903; 20080304712; 20080298649; 20080297788; 20080232653; 20080192988; 20080025580; 20080025579; 20070230754; 20070230754; 20070030475; 20060274921; 20060244947; 20060210120; 20060202028; 20060110015; 20060062438; and 20060002597.

Alcohol Measurement Modalities

Depending on the application of interest, the measurement of an analyte property can be considered in terms of two modalities. The first modality is “walk up” or “universal” and represents an analyte property determination wherein prior measurements of the sample (e.g., subject) are not used in determining the analyte property from the current measurement of interest. In the case of measuring in vivo alcohol, driving under the influence enforcement would fall into this modality as in most cases the person being tested will not have been previously measured on the alcohol measurement device. Thus, no prior knowledge of that person is available for use in the current determination of the analyte property.

The second modality is termed “enrolled” or “tailored” and represents situations where prior measurements from the sample or subject are available for use in determining the analyte property of the current measurement. An example of an environment where this modality can be applied is vehicle interlocks where a limited number of people are permitted to drive or operate a vehicle or machine. Additional information regarding embodiments of enrolled and tailored applications can be found in U.S. Pat. Nos. 6,157,041 and 6,528,809, titled “Method and Apparatus for Tailoring Spectroscopic Calibration Models”, each of which is incorporated herein by reference. In enrolled applications, the combination of the analyte property measurement with a biometric measurement can be particularly advantageous as the same spectroscopic measurement can assess if a prospective operator is authorized to use the equipment or vehicle via the biometric while the analyte property can access their fitness level (e.g., sobriety).

Alternative calibration strategies can be used in place of, or in conjunction with, the above described methods. For example, in some embodiments biometric enrollment information is acquired from each person to be measured on the device in the future. In such cases, the enrollment measurements can also be used to improve the accuracy and precision of the alcohol or substance of abuse measurement. In this scenario, the calibration spectra are mean-centered by subject (all spectra from a subject are located, the mean of those spectra is subtracted from each, and the “mean centered” spectra are returned to the spectral set). In this manner, the majority of inter-subject spectral differences caused by variations in physiology are removed from the calibration measurements and the range of spectral interferents correspondingly reduced. The centered spectra and associated analyte reference values (blood alcohol concentrations) are then presented to a multivariate analysis method such as partial least squares regression. This process is sometimes referred to as generating an “enrolled”, “generic”, or “tailored” calibration. Additional details on this approach are described in U.S. Pat. No. 6,157,041, titled “Methods and Apparatus for Tailoring Spectroscopic Calibration Models,” the disclosure of which is incorporated by reference.

In practice, once a future, post calibration, subject is enrolled on a noninvasive device their enrollment spectrum can be subtracted from subsequent measurements prior to determining the alcohol or substance of abuse concentration using the generic calibration model. Similar to the mean-centering by subject operation of the calibration spectra, the subtraction of the enrollment spectrum removes the average spectroscopic signature of the subject while preserving the signal of the attribute of interest (alcohol or substance of abuse). In some embodiments, significant performance advantages can be realized relative to the use of a non-generic calibration method.

Methods for Determining Biometric Verification or Identification from Spectroscopic Signals

Biometric identification describes the process of using one or more physical or behavioral features to identify a person or other biological entity. There are two common biometric modes: identification and verification. Biometric identification attempts to answer the question of, “do I know you?”. The biometric measurement device collects a set of biometric data from a target individual. From this information alone it assesses whether the person was previously enrolled in the biometric system. Systems that perform the biometric identification task, such as the FBI's Automatic Fingerprint Identification System (AFIS), are generally very expensive (several million dollars or more) and require many minutes to detect a match between an unknown sample and a large database containing hundreds of thousands or millions of entries. In biometric verification the relevant question is, “are you who you say you are?”. This mode is used in cases where an individual makes a claim of identity using a code, magnetic card, or other means, and the device uses the biometric data to confirm the identity of the person by comparing the target biometric data with the enrolled data that corresponds with the purported identity. The present apparatus and methods for monitoring the presence or concentration of alcohol or substances of abuse in controlled environments can use either biometric mode.

There also exists at least one variant between these two modes that is also suitable for use in the present invention. This variant occurs in the case where a small number of individuals are contained in the enrolled database and the biometric application requires the determination of only whether a target individual is among the enrolled set. In this case, the exact identity of the individual is not required and thus the task is somewhat different (and often easier) than the identification task described above. This variant might be useful in applications where the biometric system is used in methods where the tested individual must be both part of the authorized group and sober but their specific identity is not required. The term “identity characteristic” includes all of the above modes, variants, and combinations or variations thereof.

There are three major data elements associated with a biometric measurement: calibration, enrollment, and target spectral data. The calibration data are used to establish spectral features that are important for biometric determinations. This set of data consists of series of spectroscopic tissue measurements that are collected from an individual or individuals of known identity. It can be desirable to collect these data over a period of time and under conditions such that multiple spectra are collected on each individual while they span nearly the full range of physiological states that a person is expected to go through. In addition, the instrument or instruments used for spectral collection generally should also span the full range of instrumental and environmental effects that it or sister instruments are likely to see in actual use. These calibration data are then analyzed in such a way as to establish spectral wavelengths or “factors” (i.e. linear combinations of wavelengths or spectral shapes) that are sensitive to between-person spectral differences while minimizing sensitivity to within-person, instrumental (both within- and between-instruments), and environmental effects. These wavelengths or factors are then used subsequently to perform the biometric determination tasks.

The second major set of spectral data used for biometric determinations is the enrollment spectral data. The purpose of the enrollment spectra for a given subject or individual is to generate a “representation” of that subject's unique spectroscopic characteristics. Enrollment spectra are collected from individuals who are authorized or otherwise required to be recognized by the biometric system. Each enrollment spectrum can be collected over a period of seconds or minutes. Two or more enrollment measurements can be collected from the individual to ensure similarity between the measurements and rule out one or more measurements if undesirable artifacts are detected. If one or more measurements are discarded, additional enrollment spectra can be collected. The enrollment measurements for a given subject can be averaged together, otherwise combined, or stored separately. The data can be stored in an enrollment database. In some cases, each set of enrollment data can be linked with an identifier (e.g., a password or key code) for the persons on whom the spectra were measured. In the case of an identification task, the identifier can be used for record keeping purposes of who accessed the biometric system at which times. For a verification task, the identifier can be used to extract the proper set of enrollment data against which verification is performed.

The third and final major set of data used for the biometric system is the spectral data collected when a person attempts to use the biometric system for identification or verification. These data are referred to as target spectra. They can be compared to the measurements stored in the enrollment database (or subset of the database in the case of identity verification) using the classification wavelengths or factors obtained from the calibration set. In the case of biometric identification, the system compares the target spectrum to all of the enrollment spectra and reports a match if one or more of the enrolled individual's data is sufficiently similar to the target spectrum. If more than one enrolled individual matches the target, then either all of the matching individuals can be reported, or the best match can be reported as the identified person. In the case of biometric verification, the target spectrum is accompanied by an asserted identity that is collected using a magnetic card, a typed user name or identifier, a transponder, a signal from another biometric system, or other means. The asserted identity is then used to retrieve the corresponding set of spectral data from the enrollment database, against which the biometric similarity determination is made and the identity verified or denied. If the similarity is inadequate, then the biometric determination is cancelled and a new target measurement can be attempted.

In one example method of verification, principle component analysis is applied to the calibration data to generate spectral factors. These factors can then be applied to the spectral difference taken between a target spectrum and an enrollment spectrum to generate Mahalanobis distance and spectral residual magnitude values as similarity metrics. Identify is verified only if the aforementioned distance and magnitude are less than a predetermined threshold set for each. Similarly, in an example method for biometric identification, the Mahalanobis distance and spectral residual magnitude are calculated for the target spectrum relative each of the database spectra. The identity of the person providing the test spectrum is established as the person or persons associated with the database measurement that gave the smallest Mahalanobis distance and spectral residual magnitude that is less than a predetermined threshold set for each.

In an example method, the identification or verification task is implemented when a person seeks to perform an operation for which there are a limited number of people authorized (e.g., perform a spectroscopic measurement, enter a controlled facility, pass through an immigration checkpoint, etc.). The person's spectral data is used for identification or verification of the person's identity. In this preferred method, the person initially enrolls in the system by collecting one or more representative tissue spectra. If two or more spectra are collected during the enrollment, then these spectra can be checked for consistency and recorded only if they are sufficiently similar, limiting the possibility of a sample artifact corrupting the enrollment data. For a verification implementation, an identifier such as a PIN code, magnetic card number, username, badge, voice pattern, other biometric, or some other identifier can also be collected and associated with the confirmed enrollment spectrum or spectra.

In subsequent use, biometric identification can take place by collecting a spectrum from a person attempting to gain authorization. This spectrum can then be compared to the spectra in the enrolled authorization database and an identification can be made if the match to an authorized database entry is better than a predetermined threshold. The verification task is similar, but can require that the person present the identifier in addition to a collected spectrum. The identifier can then be used to select a particular enrollment database spectrum and authorization can be granted if the current spectrum is sufficiently similar to the selected enrollment spectrum. If the biometric task is associated with an operation for which only a single person is authorized, then the verification task and identification task are the same and both simplify to an assurance that the sole authorized individual is attempting the operation without the need for a separate identifier.

The biometric measurement, regardless of mode, can be performed in a variety of ways including linear discriminant analysis, quadratic discriminant analysis, K-nearest neighbors, neural networks, and other multivariate analysis techniques or classification techniques. Some of these methods rely upon establishing the underlying spectral shapes (factors, loading vectors, eigenvectors, latent variables, etc.) in the intra-person calibration database, and then using standard outlier methodologies (spectral F ratios, Mahalanobis distances, Euclidean distances, etc.) to determine the consistency of an incoming measurement with the enrollment database. The underlying spectral shapes can be generated by multiple means as disclosed herein.

First, the underlying spectral shapes can be generated based upon simple spectral decompositions (eigen analysis, Fourier analysis, etc.) of the calibration data. The second method of generating underlying spectral shapes relates to the development of a generic model as described in U.S. Pat. No. 6,157,041, titled “Methods and Apparatus for Tailoring Spectroscopic Calibration Models,” which is incorporated by reference. In this application, the underlying spectral shapes are generated through a calibration procedure performed on intra-person spectral features. The underlying spectral shapes can be generated by the development of a calibration based upon simulated constituent variation. The simulated constituent variation can model the variation introduced by real physiological or environmental or instrumental variation or can be simply be an artificial spectroscopic variation. It is recognized that other means of determining underlying shapes would be applicable to the identification and verification methods of the present invention. These methods can be used either in conjunction with, or in lieu of the aforementioned techniques.

Experimental Results: Alcohol

A clinical study was performed where ten volunteer subjects were measured in a clinical laboratory over a period of 5 days to assess tissue alcohol measurement accuracy relative to blood and breath alcohol measurements. Subjects were consented according to an IRB-approved protocol. Alcohol doses were administered to achieve peak blood alcohol concentration (BAC) values of 120 mg/dL (0.12%) assuming ingested alcohol would be completely absorbed into the bloodstream. The subjects were asked to consume the total alcohol dose within a 20-minute time period.

Baseline capillary blood, breath, and noninvasive alcohol measurements were acquired from each subject upon arrival in order to verify zero initial blood alcohol concentration. The blood measurements were acquired using a Yellow Springs Incorporated 2700 Select blood analyzer (YSI). Breath testing was accomplished using an Intoximeters EC/IR in “quick test” mode. Each subject then consumed his or her alcohol dose. Repeated cycles of blood, breath, and noninvasive measurements were then acquired to monitor alcohol concentration throughout each subject's alcohol excursion (about 10-12 minutes per cycle). A total of 372 sets of noninvasive, blood, and breath alcohol measurements were acquired from the 10 subjects in the validation study.

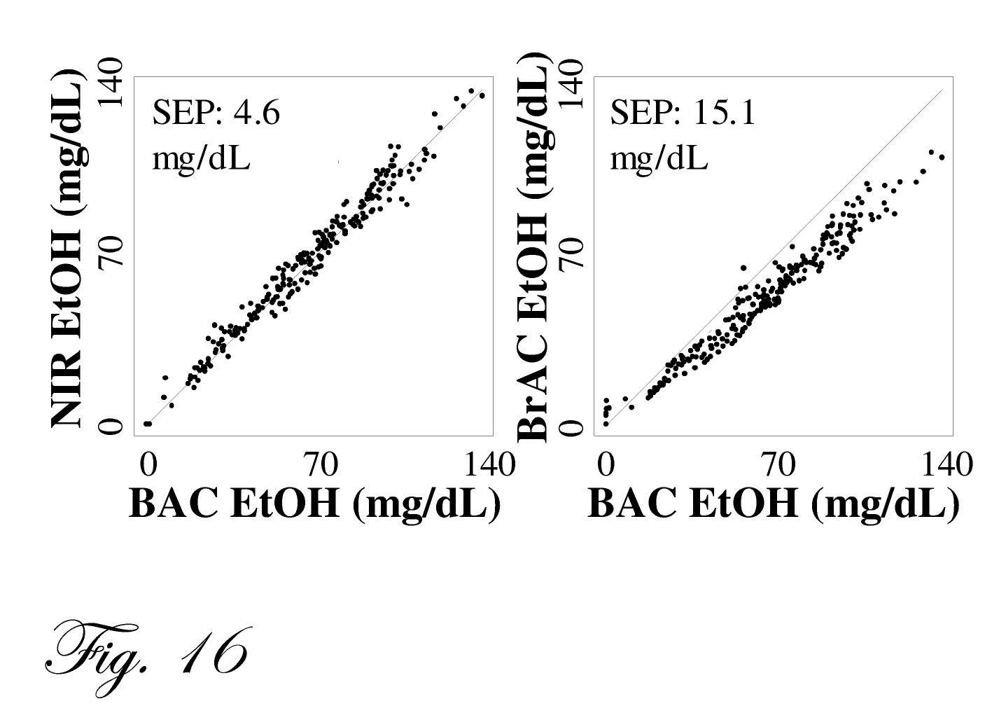

FIG. 16 shows a side-by-side comparison of the noninvasive spectroscopic alcohol measurements of the present invention versus blood (BAC) alcohol and breath (BrAC) versus blood (BAC) alcohol that were acquired from the 10 study subjects. Examination of FIG. 16 demonstrates that the breath measurements exhibit a proportional error relative to blood alcohol. This is due to the globally applied blood-breath partition coefficient of 2100 mg EtOH/dL blood per mg EtOH/dL air that relates the concentration of alcohol in expired air from the lungs to blood alcohol. The comparison of the breath and noninvasive measurements demonstrates that under identical experimental conditions the precision of the measurement of the example embodiment of the present invention is substantially equal to that of a commonly used state-of-the-art breath alcohol instrument.

Experimental Results: Biometric

An experiment was conducted to determine the viability of utilizing a methodology like those disclosed herein to verify the identification of an individual using near infrared spectroscopic measurements of skin tissue. The design of the instrumentation used was identical to that described for the experimental alcohol results discussed above. The sampling of the human tissue was done on the volar side of the forearm, consistent with the alcohol experiment. Spectra were acquired, and the recorded 4,200 to 7,200 cm⁻¹ NIR spectra converted to absorbance. The spectra consisted of two distinct sets. The first set was a calibration set comprised of 10,951 noninvasive spectroscopic measurements acquired from 209 subjects. On average, approximately 5 measurements were acquired from each subject for each of approximately 10 days. The second set of spectra was a validation set comprised of 3,159 noninvasive spectral measurements from 37 subjects. Each subject was measured approximately 85 times over a 2 month period.

The calibration spectra were processed to produce generic data as described in U.S. Pat. No. 6,157,041, titled “Methods and Apparatus for Tailoring Spectroscopic Calibration Models,” incorporated herein by reference. A PCA decomposition of these data was performed to generate 50 factors (also called latent variables, loadings, or eigenvectors) and associated scores (also called weights or eigenvalues). The validation measurements were then split into enrollment and test sets. The enrollment set was comprised of 37 spectra that were obtained by averaging the first three measurements acquired from each of the 37 validation subjects. The test set was comprised of the remaining validation spectra.

To assist in evaluating the ability of methods and apparatuses according to the present invention to correctly verify the identity of a person, the enrollment spectrum of each subject was subtracted from his or her spectra in the test set. The Mahalanobis distances of the resulting “authorized” spectral differences were then calculated using the calibration factors and scores. In order to evaluate the ability to correctly reject “intruders” (an unauthorized person who claims to be authorized in order enter or leave a controlled environment), the enrollment spectrum for a given subject was subtracted from the test spectra for the other 36 validation subjects. This was done for each validation subject in round-robin fashion in order to test all possible enrollment/test permutations. Similar to the “authorized” case, the Mahalanobis distance for each of the resulting “intruder” difference spectra was computed relative to the calibration factors and scores.

The “authorized” and “intruder” Mahalanobis distances were then used to examine the biometric performance of the spectroscopic method using multiple distance thresholds. In this framework, if the distance of a given spectral difference (whether from the “authorized” or “intruder” group) is less than the threshold distance, then the purported identity is verified. The case where an “authorized” spectral difference is below the threshold (and the identity verified) is referred to as a “True Accept” (also called a True Positive or True Admission). The case where an “authorized” spectral difference is above the threshold (the device erroneously rejects an authorized user) is referred to as a “False Reject” or “False Negative”. Similarly, a “True Reject” or “True Negative” occurs when an “intruder” distance is above the threshold and a “False Accept” occurs when an “intruder” distance is below the threshold.

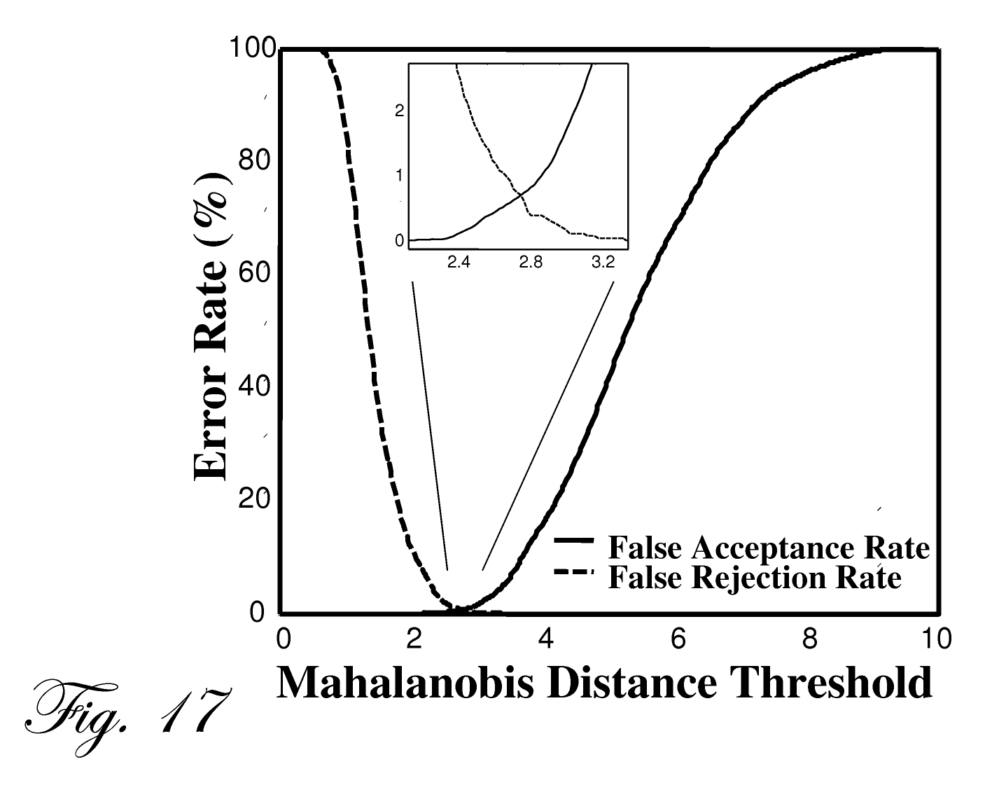

The overall performance of a technique can be compactly summarized at a given threshold by calculating the “false acceptance rate” and the “false rejection rate”. The false acceptance rate is the percentage of measurements acquired from intruders that are erroneously flagged as authorized. Conversely, the false rejection rate is the percentage of measurements acquired from authorized persons that are erroneously flagged as intruders. The threshold is a tunable variable that can be used to influence the relative security of the biometric measurement. For example, the threshold can be set to a low value (high security) that can minimize the false acceptance rate at the expense of an increase in the false rejection rate. Likewise, a low security setting would correspond to a high threshold value. In this scenario, authorized users would be rejected less frequently at the expense of an increase in intruder admission. FIG. 17 shows the false acceptance and false rejection rates at a variety of thresholds for the test data discussed above. The “equal error rate” occurs when the false acceptance and rejection rates are equal and is a common metric often used to compare biometric performance across techniques. The equal error rate for these data is approximately 0.7% demonstrating a high degree of biometric capability over an extended period of time.

Some embodiments of the present invention provide a multivariate breath tester that can accurately measure alcohol in the presence of interferents using multivariate spectroscopy. Some embodiments use multiple wavelengths, e.g., 4 or more, or 20 or more, of light. Some embodiments use inverse methods such as PLS, PCR, or MLR. Some embodiments can use dispersive systems; some can use interferometric systems. Some embodiments can report alcohol concentration and interferent presence or concentration to a user.

Some embodiments of the present invention can combine breath measurement of alcohol with tissue measurement of alcohol. Some embodiments can use near-infrared tissue measurements to measure alcohol. Some embodiments can use Raman spectroscopy to measure alcohol. Some embodiments of the present invention use a combination of breath and tissue alcohol measurement, e.g., by evaluating agreement between the two measurements as an indication of the accuracy or quality of a reported measurement.

Some embodiments of the present invention use tissue measurement of an analyte other than alcohol to evaluate the accuracy or quality of breath alcohol measurement.

Some embodiments of the present invention combine any of the preceding with a tissue biometric. Such embodiments can use a near-infrared spectroscopy biometric, a Raman spectroscopy biometric, or a visible light biometric. Some embodiments use a tissue alcohol measurement and a tissue biometric, where the tissue alcohol measurement and the tissue biometric are determined from the same spectroscopic information. Some embodiments of the present invention combine a breath alcohol measurement capability and a tissue property (e.g., alcohol, other analyte, biometric, or a combination thereof) into a single integrated instrument package.

The present invention has been described as set forth herein. It will be understood that the above description is merely illustrative of the applications of the principles of the present invention, the scope of which is to be determined by the claims viewed in light of the specification. Other variants and modifications of the invention will be apparent to those of skill in the art.

What is claimed is:

1. An apparatus for the measurement of alcohol in a breath sample including one or more interferents, comprising:
a. An optical subsystem that determines the properties of the breath sample at each of a plurality of distinct wavelengths of light;
b. An analysis subsystem that analyzes the determined properties and determines the alcohol content of the breath sample using one or more multivariate methods.

2. An apparatus as in claim 1, wherein the plurality of distinct wavelengths of light comprises at least 10 distinct wavelengths of light.

3. An apparatus as in claim 1, wherein the multivariate methods comprise at least one inverse method.

4. An apparatus as in claim 3, wherein the inverse method comprises at least one of PLS, PCR, PCA, CLS, MLR, or a combination of any of the preceding.

5. An apparatus as in claim 1, wherein the analysis system further analyzes the determined properties and determines the concentration of one or more interferents in the breath sample using one or more multivariate methods.

6. An apparatus as in claim 5, wherein the apparatus reports the alcohol concentration and the interferent concentration to a user of the apparatus.

7. An apparatus as in claim 1, wherein the optical subsystem uses one or more of the following: Raman spectroscopy, near infrared absorbance spectroscopy, near infra red reflectance spectroscopy, infra red absorbance spectroscopy, infra red reflectance spectroscopy, or a combination of any of the preceding.

8. An apparatus as in claim 1, wherein the optical subsystem comprises a solid state light source.

9. An apparatus for the measurement of alcohol, comprising:
a. A breath alcohol subsystem that measures alcohol based on breath;
b. A tissue analyte subsystem that measures an analyte based on one or more optical tissue measurements;
c. A display subsystem that communicates to a user at least one of: results from each of the breath alcohol subsystem and the tissue analyte subsystem, an integrated result determined from a combination of the results of the breath alcohol subsystem and the tissue analyte subsystem, an indication that the results of the breath alcohol subsystem and the tissue analyte subsystem indicate that an accurate alcohol measurement was not obtained.

10. An apparatus as in claim 9, wherein the breath alcohol subsystem comprises an apparatus as in claim 1.

11. An apparatus as in claim 9, wherein the tissue analyte subsystem measures alcohol in tissue.

12. An apparatus as in claim 9, wherein the tissue analyte subsystem measures a substance in tissue whose presence indicates reduced accuracy of the breath alcohol subsystem.

13. An apparatus as in claim 9, wherein the tissue analyte subsystem measures the rate of change of alcohol in tissue.

14. An apparatus as in claim 9, wherein the tissue analyte subsystem measures one or more substances of abuse.

15. An apparatus for the measurement of alcohol, comprising:
a. A breath alcohol subsystem that measures alcohol based on breath;
b. A tissue biometric subsystem that determines one or more identity characteristics based on optical tissue measurements;
c. A display subsystem that communicates to a user at least one of: a result from the breath alcohol subsystem and the one or more identity characteristics, a result from the results of the breath alcohol subsystem only if the one or more identity characteristics is acceptable, an indication that an action is allowed only if the result from the breath alcohol subsystem and the result from the tissue biometric subsystem both indicate acceptance.

16. An apparatus as in claim 15, wherein the breath alcohol subsystem is an apparatus as in claim 1.

17. An apparatus as in claim 15, wherein the tissue biometric subsystem comprises one or more of: a near-infrared biometric subsystem, a Raman spectroscopic biometric subsystem, a visible light biometric subsystem.

18. An apparatus as in claim 15, further comprising a tissue analyte subsystem that measures an analyte based on one or more optical tissue measurements.

19. An apparatus as in claim 18, wherein the breath alcohol subsystem is an apparatus as in claim 1.

20. An apparatus as in claim 1, further comprising one or more of a biometric subsystem, a tissue biometric subsystem, an alcohol measurement subsystem based on a property other than breath, a tissue alcohol measurement subsystem, a substance of abuse measurement subsystem, or a combination of any of the preceding.
